(19) United States
(12) Patent Application Publication
(10) Pub. No.: US 2007/0178452 A1
(43) Pub. Date: Aug. 2, 2007

(54) Method for analyzing a nucleic acid

(76) Inventors: Pascal Bouffard, Danbury, CT (US); John L. Herrmann, Guilford, CT (US); Chunli Huang, New Haven, CT (US); Michael Jeffers, Branford, CT (US); Jingfang Ju, Orange, CT (US); Luca Rastelli, Guilford, CT (US); Juliette Shimkets, Guilford, CT (US); Jan Simons, New Haven, CT (US); Bruce Taillon, Middletown, CT (US)

Correspondence Address: Jenell Lawson;Intellectual Property, CuraGen Corporation, 555 Long Wharf Drive, New Haven, CT, 06551, US

(21) Appl. No.: 10/277,951

(22) Filed: Oct. 21, 2002

Related U.S. Application Data
(63) Continuation-in-part of application No. 09/862,101, filed on May 21, 2001, now abandoned.
(60) Provisional application No. 60/205,385, filed on May 19, 2000.
(60) Provisional application No. 60/265,394, filed on Jan. 31, 2001.
(60) Provisional application No. 60/282,982, filed on Apr. 11, 2001.

(51) Int. Cl. C12Q 1/68; G06F 19/00

(52) U.S. Cl. 435/6; 702/20

(57) ABSTRACT

Disclosed is a method in which DNA sequences derived from microsome-associated mRNA sequences in a mixed sample or in an arrayed single sequence clone can be determined and classified without sequencing. The methods make use of information on the presence of carefully chosen target subsequences, typically of length from 4 to 8 base pairs, and preferably the length between target subsequences in a sample DNA sequence together with DNA sequence databases containing lists of sequences likely to be present in the sample to determine a sample sequence. The preferred method uses restriction endonucleases to recognize target subsequences and cut the sample sequence. Then carefully chosen recognition moieties are ligated to the cut fragments, the fragments amplified, and the experimental observation made. Polymerase chain reaction (PCR) is the preferred method of amplification. Another embodiment of the invention uses information on the presence or absence of carefully chosen target subsequences in a single sequence clone together with DNA sequence databases to determine the clone sequence. Computer implemented methods are provided to analyze the experimental results and to determine the sample sequences in question and to carefully choose target subsequences in order that experiments yield a maximum amount of information

### RELATED APPLICATIONS

This application is a continuation-in-part application of U.S. Ser. No. 09/862,101, filed May 21, 2001, which claims priority to U.S. Ser. No. 60/205,385, filed May 19, 2000; U.S. Ser. No. 60/265,394, filed Jan. 31, 2001; and U.S. Ser. No. 60/282,982, filed Apr. 11, 2001, and claims priority to U.S. Ser. No. 60/348,907, filed Oct. 22, 2001; and U.S. Ser. No. 60/347,762, filed Jan. 11, 2002. These applications are incorporated herein by reference in their entireties.

### FIELD OF THE INVENTION

The invention relates to nucleic acid sequence classification, identification, or quantification.

### BACKGROUND OF THE INVENTION

Gene expression can be regulated at multiple levels, such as transcription, mRNA processing, mRNA transport, mRNA stability, translation initiation, translation elongation and post-translational modification. Currently available quantitative gene expression analyses have mostly been performed at the transcriptional level by measuring steady-state levels of mRNAs. While these methods provide a measure of the change or difference in gene transcription it does not provide a measure of gene expression regulation occurring at the translational (or protein production) level.

Secreted proteins are characterized by the presence of a hydrophobic signal peptide at the amino terminus of the protein. The hydrophobic signal sequence is typically from about 16 to about 30 amino acids long and contains one or more positively charged amino acid residues near its N-terminus, followed by a continuous stretch of 6-12 hydrophobic residues. Signal peptides from various secreted proteins have otherwise no sequence homology. The presence of a hydrophobic signal peptide at the amino terminus of a protein mediates its association with the rough endoplasmic reticulum (ER), which in turn mediates its secretion from the cell.

Peptides or proteins having a signal peptide associated with the endoplasmic reticulum are secreted by the following mechanism. Protein synthesis begins on free ribosomes. When the elongating peptide is about 70 amino acids long, the signal peptide is recognized by a particle, termed a “signal recognition particle” or “SRP”, which in turn is capable of interacting with a receptor, termed “SRP receptor”, located on the ER. Thus, growing peptides having a signal peptide are targeted to the ER, where peptide synthesis continues on the rough ER. At some point during the protein synthesis or after the protein synthesis is completed, the protein is translocated across the ER membrane into the ER lumen, where the signal peptide is cleaved off. There the protein can be post-translationally modified, e.g., glycosylated. Whether post-translationally modified or not, the protein can then be directed to the appropriate cellular compartment, e.g., secreted outside the cell.

### SUMMARY OF THE INVENTION

The invention provides methods for quantifying gene expression regulation that occurs via changes in translation efficiency. The invention is based at least in part on the observation that nucleic acid molecules encoding secreted proteins can be cloned from RNA that is isolated from microsomes. In one embodiment, actively translated mRNAs are identified first through isolation of a microsomal fraction, e.g., a subcellular fraction containing microsomes that contain ribosomes and an mRNA species undergoing active translation. The mRNA is converted into CDNA and analyzed on an open expression analysis platform, e.g. an analysis platform that does not require a priori knowledge of sequence information, for quantitation and gene identification. Levels of actively translated mRNAs can compared to total mRNA levels or different translated mRNA populations can be compare under different conditions. These comparisons reveal fundamental differences between regulation of gene expression at the transcriptional and translational levels. This information can be used to identify genes and gene products of fundamental importance.

The invention also provides a method for enriching a population of RNA molecules in those RNA molecules encoding a secreted protein or a protein having a signal peptide. The enrichment of the RNA population with RNA molecules containing a signal sequence can be of a factor of about 2 to about 5, of about 5 to about 10, at least about 100, at least about 10³, at least about 10⁴, at least about 10⁵, at least about 10⁶, at least about 10⁷ or at least about 10⁸.

In one aspect the invention relates to a method for identifying, classifying, or quantifying one or more nucleic acids in a sample having a plurality of nucleic acids having different nucleotide sequences, the method including the steps of: (a) providing a cDNA sample prepared from a population of microsomes; (b) probing the sample with one or more recognition means, each recognition means recognizing a different target nucleotide subsequence or a different set of target nucleotide subsequences; (c) generating one or more output signals from the sample probed by the recognition means, each output signal being produced from a nucleic acid in the sample by recognition of one or more target nucleotide subsequences in the nucleic acid by the recognition means and including a representation of (i) the length between occurrences of target nucleotide subsequences in the nucleic acid, and (ii) the identities of the target nucleotide subsequences in the nucleic acid or the identities of the sets of target nucleotide subsequences among which are included the target nucleotide subsequences in the nucleic acid; and (d) searching a nucleotide sequence database to determine sequences that are predicted to produce or the absence of any sequences that are predicted to produce the one or more output signals produced by the nucleic acid, the database including a plurality of known nucleotide sequences of nucleic acids that may be present in the sample, a sequence from the database being predicted to produce the one or more output signals when the sequence from the database has both (i) the same length between occurrences of target nucleotide subsequences as is represented by the one or more output signals, and (ii) the same target nucleotide subsequences as are represented by the one or more output signals, or target nucleotide subsequences that are members of the same sets of target nucleotide subsequences represented by the one or more output signals, whereby the one or more nucleic acids in the sample are identified, classified, or quantified.

In an embodiment of the invention, each of the recognition means recognizes one target nucleotide subsequence, and where a sequence from the database is predicted to produce a particular output signal when the sequence from the database has both the same length between occurrences of target nucleotide subsequences as is represented by the output signal and the same target nucleotide subsequences as represented by the particular output signal.

In a related embodiment, the database includes substantially all the known expressed sequences of the plant, single celled animal, multicellular animal, bacterium, virus, fungus, or yeast.

In another embodiment of the invention, each recognition means recognizes a set of target nucleotide subsequences, and wherein a sequence from the database is predicted to produce a particular output signal when the sequence from the database has both the same length between occurrences of target nucleotide subsequences as is represented by the particular output signal, and the target nucleotide subsequences are members of the sets of target nucleotide subsequences represented by the particular output signal.

In a further embodiment of the invention, the method also includes dividing the sample of nucleic acids into a plurality of portions and performing the method individually on a plurality of the portions, wherein a different one or more recognition means are used with each portion.

In yet another embodiment of the invention, the quantitative abundances of nucleic acids in the sample are determined from the quantitative levels of the output signals produced by the nucleic acids.

In another embodiment, the cDNA is prepared from a plant, a single celled animal, a multicellular animal, a bacterium, a virus, a fungus, or a yeast. In another embodiment, the CDNA is prepared from a mammal. In a related embodiment, the mammal is a human. In another related embodiment, the CDNA is of total cellular RNA or total cellular poly(A) RNA.

In certain embodiments, the recognition means are one or more restriction endonucleases whose recognition sites are the target nucleotide subsequences, and wherein the step of probing comprises digesting the sample with the one or more restriction endonucleases into fragments and ligating double stranded adapter DNA molecules to the fragments to produce ligated fragments, each the adapter DNA molecule comprising (i) a shorter stand having no 5′ terminal phosphates and consisting of a first and second portion, the first portion at the 5′ end of the shorter strand and being complementary to the overhang produced by one of the restriction endonucleases, and (ii) a longer strand having a 3′ end subsequence complementary to the second portion of the shorter strand; and wherein the step of generating further comprises melting the shorter strand from the ligated fragments, contacting the ligated fragments with a DNA polymerase, extending the ligated fragments by synthesis with the DNA polymerase to produce blunt-ended double stranded DNA fragments, and amplifying the blunt-ended fragments by a method comprising contacting the blunt-ended fragments with the DNA polymerase and primer oligodeoxynucleotides, the primer oligodeoxynucleotides comprising a hybridizable portion of the sequence of the longer strand of the adapter nucleic acid molecule, and the contacting being at a temperature not greater than the melting temperature of the primer oligodeoxynucleotide from a strand of the blunt-ended fragments complementary to the primer oligodeoxynucleotide and not less than the melting temperature of the shorter strand of the adapter nucleic acid molecule from the blunt-ended fragments.

In another embodiment of the invention, the recognition means are one or more restriction endonucleases whose recognition sites are the target nucleotide subsequences, and wherein the step of probing further comprises digesting the sample into fragments with the one or more restriction endonucleases. In a related embodiment, the method of the invention further includes (a) identifying a fragment of a nucleic acid in the sample which generates the one or more output signals; and (b) recovering the fragment. In another related embodiment, the output signals generated by the recovered fragment are not predicted to be produced by a sequence in the nucleotide sequence database.

In another embodiment of the invention, the method also includes using at least a hybridizable portion of the recovered fragment as a hybridization probe to bind to a nucleic acid.

In another embodiment, the step of generating further comprises after the digesting: removing from the sample both nucleic acids which have not been digested and nucleic acid fragments resulting from digestion at only a single terminus of the fragments. In a related embodiment, the method includes that, prior to digesting, the nucleic acids in the sample are each bound at one terminus to a biotin molecule, and the removing is carried out by a method which comprises contacting the nucleic acids in the sample with streptavidin or avidin affixed to a solid support.

In another embodiment, prior to digestion, the nucleic acids in the sample are each bound at one terminus to a hapten molecule, and the removing is carried out by a method which comprises contacting the nucleic acids in the sample with an anti-hapten antibody affixed to a solid support.

In yet another embodiment, the digesting with the one or more restriction endonucleases leaves single-stranded nucleotide overhangs on the digested ends.

In a further embodiment, the invention includes a step of probing that includes hybridizing double-stranded adapter nucleic acids with the digested sample fragments, each the double-stranded adapter nucleic acid having an end complementary to the overhang generated by a particular one of the one or more restriction endonucleases, and ligating with a ligase a strand of the double-stranded adapter nucleic acids to the 5′ end of a strand of the digested sample fragments to form ligated nucleic acid fragments. In a related embodiment, the digesting with the one or more restriction endonucleases and the ligating are carried out in the same reaction medium. In a further related embodiment, the digesting and the ligating comprises incubating the reaction medium at a first temperature and then at a second temperature, wherein the one or more restriction endonucleases are more active at the first temperature than the second temperature and the ligase is more active at the second temperature than the first temperature. In another related embodiment, the incubating at the first temperature and the incubating at the second temperature are performed repetitively.

In another embodiment, the step of probing further comprises prior to the digesting: removing terminal phosphates from DNA in the sample by incubation with an alkaline phosphatase. In a related embodiment, the alkaline phosphatase is heat labile and is heat inactivated prior to the digesting.

In another embodiment, the generating step comprises amplifying the ligated nucleic acid fragments.

In another embodiment, the amplifying step is carried out by use of a nucleic acid polymerase and primer nucleic acid strands, the primer nucleic acid strands comprising a hybridizable portion of the sequence of the strands ligated to the sample fragments. In a related embodiment, the primer nucleic acid strands have a G+C content of between 40% and 60%.

In yet another embodiment, each of the double-stranded adapter nucleic acid comprises a shorter strand hybridized to a longer strand, wherein the longer strand is the strand of the double-stranded adapter nucleic acid that becomes ligated to the digested sample fragments, wherein each the shorter strand is complementary both to one of the single-stranded nucleotide overhangs and to one of the longer strands, and the generating step comprises prior to the amplifying step the melting of the shorter strand from the ligated fragments, contacting the ligated fragments with a DNA polymerase, extending the ligated fragments by synthesis with the DNA polymerase to produce blunt-ended double stranded DNA fragments, and wherein the primer nucleic acid strands comprise a hybridizable portion of the sequence of the longer strands. In certain embodiments, each the double-stranded adapter nucleic acid comprises a shorter strand hybridized to a longer strand, wherein the longer strand is the strand of the double-stranded adapter nucleic acid that becomes ligated to the digested sample fragments, wherein each the shorter strand is complementary both to one of the single-stranded nucleotide overhangs and to one of the longer strands, and the generating step comprises prior to the amplifying step the melting of the shorter strand from the ligated fragments, contacting the ligated fragments with a DNA polymerase, extending the ligated fragments by synthesis with the DNA polymerase to produce blunt-ended double stranded DNA fragments, and wherein the primer nucleic acid strands comprise the sequence of the longer strands.

In another embodiment of the invention, in the amplifying step the primer nucleic acid strands are annealed to the ligated nucleic acid fragments at a temperature that is less than the melting temperature of the primer nucleic acid strands from strands complementary to the primer nucleic acid strands but greater than the melting temperature of the shorter adapter strands from the blunt-ended fragments.

In another embodiment, the primer nucleic acid strands further comprise at the 3′ end of and contiguous with the longer strand sequence, the sequence of the portion of the restriction endonuclease recognition site remaining on a nucleic acid fragment terminus after digestion by the restriction endonuclease. In a related embodiment, each the primer nucleic acid strand further comprises at its 3′ end one or more additional nucleotides 3′ to and contiguous with the sequence of the portion of the restriction endonuclease recognition site remaining on a nucleic acid fragment after digestion by the restriction endonuclease, whereby the ligated nucleic acid fragment amplified is that comprising the remaining portion of the restriction endonuclease recognition site contiguous to the one or more additional nucleotides. In another related embodiment, the primer nucleic acid strands are detectably labeled, such that the primer nucleic acid strands comprising a particular the one or more additional nucleotides can be detected and distinguished from the primer nucleic acid strands comprising a different the one or more additional nucleotides.

In another embodiment of the invention, the recognition means comprise oligomers of nucleotides, universal nucleotides, nucleotide-mimics, or a combination of nucleotides, universal nucleotides, and nucleotide-mimics, the oligomers being hybridizable with the target nucleotide subsequences. In a related embodiment, the step of generating comprises amplifying with a nucleic acid polymerase and with primers, the sequence of the primers comprising (i) the sequence of the oligomers, and (ii) an additional subsequence 5′ to the sequence of the oligomers. In certain embodiments, the invention further includes the steps of (a) identifying a fragment of a nucleic acid in the sample which generates the one or more output signals; and (b) recovering the fragment. In related embodiments, the one or more output signals generated by the recovered fragment are not predicted to be produced by any sequence in the nucleotide database.

In another embodiment, the invention further includes using at least a hybridizable portion of the recovered fragment as a hybridization probe to bind to a nucleic acid.

In another embodiment, the one or more output signals further comprise a representation of whether an additional target nucleotide subsequence is present in the nucleic acid in the sample between the occurrences of target nucleotide subsequences. In a related embodiment, the additional target nucleotide subsequence is recognized by a method including contacting nucleic acids in the sample with oligomers of nucleotides, nucleotide-mimics, or mixed nucleotides and nucleotide-mimics, which are hybridizable with the additional target nucleotide subsequence.

In another embodiment, the step of generating comprises generating the one or more output signals only when an additional target nucleotide subsequence is not present in the nucleic acid in the sample between the occurrences of target nucleotide subsequences, and wherein a sequence from the sequence database is predicted to produce the one or more output signals when the sequence from the database (i) has the same length between occurrences of target nucleotide subsequences as is represented by the one ore more output signals, (ii) has the same target nucleotide subsequences as are represented by the one or more output signals, or target nucleotide subsequences that are members of the same sets of target nucleotide subsequences as are represented by the one or more output signals and (iii) does not contain the additional target nucleotide subsequence between occurrences of the target nucleotide subsequences.

In yet another embodiment, the step of generating comprises amplifying nucleic acids in the sample, and wherein the additional target nucleotide subsequence is recognized by a method including contacting nucleic acids in the sample with (a) oligomers of nucleotides, nucleotide-mimics, or mixed nucleotides and nucleotide-mimics, which hybridize with the additional target nucleotide subsequence and disrupt the amplifying step; or (b) restriction endonucleases which have the additional target nucleotide subsequence as a recognition site and digest the nucleic acids in the sample at the recognition site.

In another embodiment, the step of generating further comprises separating nucleic acid fragments by length. In a related embodiment, step of generating further comprises detecting the separated nucleic acid fragments. In other related embodiments the abundance of a nucleic acid including a particular nucleotide sequence in the sample is determined from the level of the one or more output signals produced by the nucleic acid that are predicted to be produced by the particular nucleotide sequence.

In another embodiment, the detecting is carried out by a method including staining the fragments with silver, labeling the fragments with a DNA intercalating dye, or detecting light emission from a fluorochrome label on the fragments.

In another embodiment of the invention, the representation of the length between occurrences of target nucleotide subsequences is the length of fragments determined by the separating and detecting steps. In a related embodiment, the separating is carried out by use of liquid chromatography or mass spectrometry. In an alternative related embodiment, the separating is carried out by use of electrophoresis. In a further related embodiment, the electrophoresis is carried out in a gel arranged in a slab or arranged in a capillary using a denaturing or non-denaturing medium.

In another embodiment of the invention, a predetermined one or more nucleotide sequences in the database are of interest, and wherein the target nucleotide subsequences are such that the sequences of interest are predicted to produce at least one output signal that is not predicted to be produced by other nucleotide sequences in the database. In a related embodiment, the nucleotide sequences of interest are a majority of the sequences in the database.

Another aspect of the present invention relates to a method for identifying or classifying a nucleic acid in a microsomal sample including a plurality of nucleic acids having different nucleotide sequences, the method including: (a) providing a nucleic acid; (b) probing the nucleic acid with a plurality of recognition means, each recognition means recognizing a target nucleotide subsequence or a set of target nucleotide subsequences, in order to produce an output set of signals, each signal of the output set representing whether the target nucleotide subsequence or one of the set of target nucleotide subsequences is present in the nucleic acid; and (c) searching a nucleotide sequence database, the database including a plurality of known nucleotide sequences of nucleic acids that may be present in the sample, for sequences predicted to produce the output set of signals, a sequence from the database being predicted to produce an output set of signals when the sequence from the database (i) comprises the same target nucleotide subsequences represented as present, or comprises target nucleotide subsequences that are members of the sets of target nucleotide subsequences represented as present by the output set of signals, and (ii) does not comprise the target nucleotide subsequences not represented as present or that are members of the sets of target nucleotide subsequences not represented as present by the output set of signals, whereby the nucleic acid is identified or classified.

Another aspect of the present invention relates to a method for identifying, classifying, or quantifying DNA molecules in a sample of DNA molecules with a plurality of nucleotide sequences, the method including the steps of: (a) providing a cDNA sample synthesized from microsomal RNA molecules; (b) digesting the sample with one or more restriction endonucleases, each the restriction endonuclease recognizing a subsequence recognition site and digesting DNA to produce fragments with 3′ overhangs; (c) contacting the fragments with shorter and longer oligodeoxynucleotides, each the longer oligodeoxynucleotide consisting of a first and second contiguous portion, the first portion being a 3′ end subsequence complementary to the overhang produced by one of the restriction endonucleases, each the shorter oligodeoxynucleotide complementary to the 3′ end of the second portion of the longer oligodeoxynucleotide stand; (d) ligating the longer oligodeoxynucleotides to the DNA fragments to produce a ligated fragments and removing the shorter oligodeoxynucleotides from the ligated DNA fragments; (e) extending the ligated DNA fragments by synthesis with a DNA polymerase to form blunt-ended double stranded DNA fragments; (f) amplifying the double stranded DNA fragments by use of a DNA polymerase and primer oligodeoxynucleotides to produce amplified DNA fragments, each the primer oligodeoxynuclcotide having a sequence including that of a longer oligodeoxynucleotide; (g) determining the length of the amplified DNA fragments; and (h) searching a DNA sequence database, the database including a plurality of known DNA sequences that may be present in the sample, for sequences predicted to produce one or more of the fragments of determined length, a sequence from the database being predicted to produce a fragment of determined length when the sequence from the database comprises recognition sites of the one or more restriction endonucleases spaced apart by the determined length, whereby DNA sequences in the sample are identified, classified, or quantified.

Another aspect of the invention relates to a method of detecting one or more differentially expressed genes in an in vitro cell exposed to an exogenous factor relative to an in vitro cell not exposed to the exogenous factor including: (a) performing the method of claim 1 wherein the plurality of nucleic acids comprises CDNA of RNA isolated from a microsome of the in vitro cell exposed to the exogenous factor; (b) performing the method of claim 1 wherein the plurality of nucleic acids comprises CDNA of RNA isolated from a microsome of the in vitro cell not exposed to the exogenous factor; and (c) comparing the identified, classified, or quantified cDNA of the in vitro cell exposed to the exogenous factor with the identified, classified, or quantified CDNA of the in vitro cell not exposed to the exogenous factor, whereby differentially expressed genes are identified, classified, or quantified.

Another aspect of the present invention relates to a method of detecting one or more differentially expressed genes in a diseased tissue relative to a tissue not having the disease including: (a) performing the method of claim 1 wherein the plurality of nucleic acids comprises cDNA of RNA of the diseased tissue, such that one or more cDNA molecules are identified, classified, and/or quantified; (b) performing the method of claim 1 wherein the plurality of nucleic acids comprises cDNA of RNA of the tissue not having the disease, such that one or more cDNA molecules are identified, classified, and/or quantified; and (c) comparing the identified, classified, and/or quantified cDNA molecules of the diseased tissue with the identified, classified, and/or quantified cDNA molecules of the tissue not having the disease, whereby differentially expressed cDNA molecules are detected. In an embodiment of this invention, the step of comparing further comprises determining cDNA molecules which are reproducibly expressed in the diseased tissue or in the tissue not having the disease and further determining which of the reproducibly expressed cDNA molecules have significant differences in expression between the tissue having the disease and the tissue not having the disease. In a related embodiment, the determining CDNA molecules which are reproducibly expressed and the significant differences in expression of the cDNA molecules in the diseased tissue and in the tissue not having the disease are determined by a method including applying statistical measures.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

FIG. 1 is a schematic diagram of polysomal sample preparation and quantitative expression analysis.

FIG. 2 is an optical density profile of sucrose gradients loaded with extracts of untreated MG-63 cells (left panel) or extracts of IL-1α treated MG-63 cells (right panel).

FIG. 3 is a trace replication profile for translational initiation factor 4B from treated MG-63 cells (Set A) and untreated MG-63 cells (Set B).

FIG. 4 is a trace replication profile for human phosphatase 2A from IL-1α treated MG-63 cells (Set A) and untreated MG-63 cells (Set B).

FIG. 5 is a Western immunoblot of CAML in extracts from untreated MG-63 cells (Lane 1) and extracts from IL-1α treated MG-63 cells (Lane 2).

FIG. 6 is a Western immunoblot of the rough ER marker protein calnexin in sucrose gradient fractionated lysate from human melanoma cells.

### DETAILED DESCRIPTION OF THE INVENTION

The invention provides methods for identifying genes being actively transcribed in a population of cells. It has been established that translational regulation plays a critical role in many biological process, e.g., in cell cycle progression under normal and stress conditions (Sheikh et al., Oncogene 18 6121-28, 1999). Translational regulation provides the cell with a more precise, immediate and energy-efficient way to control the expression of a given protein. Translational regulation can induce rapid changes in protein synthesis without the need for transcriptional activation and subsequent mRNA processing steps. In addition, translational control also has the advantage of being readily reversible, providing the cell with great flexibility in responding to various cytotoxic stresses. Therefore, it is useful to know not just the levels of individual mRNAs, but also to what extent they are being translated into their corresponding proteins. The simultaneous monitoring of cellular mRNA levels and the translation state of all mRNAs provides a more complete description of gene expression.

The endoplasmic reticulum (ER) of eukaryotic cells provides the cells with a mechanism for separating newly synthesized molecules that belong to the cytoplasm from those that do not. Lipids, proteins and complex carbohydrates destined for transportation to the Golgi apparatus, to the plasma membrane, to lysosomes, or to the cell exterior are all synthesized in association with the ER. Association of proteins with rough ER is mediated through the presence of a hydrophobic signal peptide at the amino terminus of the protein.

The ER has two functionally and structurally distinct regions: the rough endoplasmic reticulum, which is covered with ribosomes on the cytoplasmic side of the membrane and the smooth endoplasmic reticulum, which lacks ribosomes. The rough endoplasmic ribosome is involved in the synthesis of secretory proteins, integral, ER, Golgi, and plasma-membrane proteins, glycoproteins and lysosome proteins. Though all nucleated cells, except sperm cells, have ER, the amount of rough ER varies from one cell type to another, depending of the function of the cell. For example, a cell specialized in protein secretion, such as a pancreatic acinar cell and antibody secreting plasma cell, or a cell undergoing extensive membrane synthesis, e.g., an immature egg or a retinal rod cell, are particularly rich in rough ER. The smooth ER is not involved in protein synthesis.

Upon disruption of a tissue or cells by homogenization, the ER is fragmented into many smaller (about 100 nm diameter) closed vesicles called “microsomes”, which are relatively easy to purify. Microsomes derived from the rough ER are covered with ribosomes on the outside of the microsome and are termed “rough microsomes”. Such a tissue or cell homogenate also contains many vesicles of a size similar to the rough microsomes, but which do not contain ribosomes on their surface. Such smooth microsomes are derived in part from the smooth portions of the ER and in part from vesiculated fragments of plasma membranes, Golgi apparatus, and mitochondria. Rough microsomes can be separated from smooth microsomes, e.g., by sucrose gradient centrifugation. In fact, smooth microsomes have a low density and stop sedimenting and float at a low sucrose concentration, whereas rough microsomes have a high density and stop sedimenting and float at high sucrose concentration. (See, e.g., U.S. Pat. No. 6,066,460).

The present invention provides a method and reagents for isolating a nucleic acid encoding a secreted protein or a protein having a signal peptide, by isolating an RNA molecule from a microsomal fraction or other ER preparation. In a preferred embodiment, the protein having a signal peptide is a secreted protein. The protein can also be an integral protein, an ER protein, a Golgi protein, a plasma-membrane protein, a glycoprotein, or a lysosome protein.

Recent studies that combine polysomal isolation and micro-array based CDNA chip analysis demonstrated the feasibility and value of performing high-throughput analysis of the mRNA translation state (Zong et al., Proc. Natl. Acad. Sci. USA; 96: 10632-36, 1999; Johannes et al., Proc. Natl. Acad. Sci. USA 96: 13118-23, 1999).

For example, RNA binding proteins are reported to be regulated at the translational level and can be important targets for drug development (Chu et al., Stem Cells 14: 41-6, 1996). The methods described combine polysomal isolation with an open high-throughput quantitative mRNA analysis detection platform, which simultaneously can detect and identify every existing mRNA was used to prepare samples for analysis by an open high-throughput mRNA expression analysis technology (Shimkets et al., Nature Biotech 17:798-803, 1999).

Any art-recognized method for isolating polysomal RNA can be used. Isolation methods are discussed (e.g., Ruan et al. In: Analysis of mRNA Formation and Function, ed. Richter, J. D. (Academic, New York), 1997, pp., 305-321). Methods for isolating microsomes and microsomal RNA are discussed in Example 3.

A preferred method of measuring gene expression from microsomal RNA is the mRNA profiling technique described in U.S. Pat. No. 5, 871,697, W097/15690, and Shimkets et al., Nature Biotech 17:798-803, 1999. This method permits high-throughput reproducible detection of most expressed sequences with a sensitivity of greater than I part in 100,000. Gene identification by database query of a restriction endonuclease fingerprint, confirmed by competitive PCR using gene-specific oligonucleotides, facilitates gene discovery by minimizing isolation procedures.

It is an object of this invention to provide methods for rapid, economical, quantitative, and precise determination or classification of cDNA sequences generated from mRNA molecules recovered from ribosomes, e.g., polysomes or microsomes. The sequences can be provided in either arrays of single sequence clones or mixtures of sequences such as can be derived from tissue samples, without actually sequencing the DNA. Thereby, the deficiencies in the background arts just identified are solved. This object is realized by generating a plurality of distinctive and detectable signals from the DNA sequences in the sample being analyzed. Preferably, all the signals taken together have sufficient discrimination and resolution so that each particular DNA sequence in a sample may be individually classified by the particular signals it generates, and with reference to a database of DNA sequences possible in the sample, individually determined. The intensity of the signals indicative of a particular DNA sequence depends quantitatively on the amount of that DNA present. Alternatively, the signals together can classify a predominant fraction of the DNA sequences into a plurality of sets of approximately no more than two to four individual sequences.

It is a further object that the numerous signals be generated from measurements of the results of as few a number of recognition reactions as possible, preferably no more than approximately 5-400 reactions, and most preferably no more than approximately 20-50 reactions. Rapid and economical determinations would not be achieved if each DNA sequence in a sample containing a complex mixture required a separate reaction with a unique probe. Preferably, each recognition reaction generates a large number of or a distinctive pattern of distinguishable signals, which are quantitatively proportional to the amount of the particular DNA sequences present. Further, the signals are preferably detected and measured with a minimum number of observations, which are preferably capable of simultaneous performance.

The signals are preferably optical, generated by fluorochrome labels and detected by automated optical detection technologies. Using these methods, multiple individually labeled moieties can be discriminated even though they are in the same filter spot or gel band. This permits multiplexing reactions and parallelizing signal detection. Alternatively, the invention is easily adaptable to other labeling systems, for example, silver staining of gels. In particular, any single molecule detection system, whether optical or by some other technology such as scanning or tunneling microscopy, would be highly advantageous for use according to this invention as it would greatly improve quantitative characteristics.

According to this invention, signals are generated by detecting the presence (hereinafter called “hits”) or absence of short DNA subsequences (hereinafter called “target” subsequences) within a nucleic acid sequence of the sample to be analyzed. The presence or absence of a subsequence is detected by use of recognition means, or probes, for the subsequence. The subsequences are recognized by recognition means of several sorts, including but not limited to restriction endonucleases (“REs”), DNA oligomers, and peptide nucleic acid (“PNA”) oligomers. REs recognize their specific subsequences by cleavage thereof; DNA and PNA oligomers recognize their specific subsequences by hybridization methods. The preferred embodiment detects not only the presence of pairs of hits in a sample sequence but also include a representation of the length in base pairs between adjacent hits. This length representation can be corrected to true physical length in base pairs upon removing experimental biases and errors of the length separation and detection means. An alternative embodiment detects only the pattern of hits in an array of clones, each containing a single sequence (“single sequence clones”).

The generated signals are then analyzed together with DNA sequence information stored in sequence databases in computer implemented experimental analysis methods of this invention to identify individual genes and their quantitative presence in the sample.

The target subsequences are chosen by further computer implemented experimental design methods of this invention such that their presence or absence and their relative distances when present yield a maximum amount of information for classifying or determining the DNA sequences to be analyzed. Thereby it is possible to have orders of magnitude fewer probes than there are DNA sequences to be analyzed, and it is further possible to have considerably fewer probes than would be present in combinatorial libraries of the same length as the probes used in this invention. For each embodiment, target subsequences have a preferred probability of occurrence in a sequence, typically between 5% and 50%. In all embodiments, it is preferred that the presence of one probe in a DNA sequence to be analyzed is independent of the presence of any other probe.

Preferably, target subsequences are chosen based on information in relevant DNA sequence databases that characterize the sample. A minimum number of target subsequences may be chosen to determine the expression of all genes in a tissue sample (“tissue mode”). Alternatively, a smaller number of target subsequences may be chosen to quantitatively classify or determine only one or a few sequences of genes of interest, for example oncogenes, tumor suppressor genes, growth factors, cell cycle genes, cytoskeletal genes, etc (“query mode”).

A preferred embodiment of the invention, named quantitative expression analysis (“QEA”), produces signals including target subsequence presence and a representation of the length in base pairs along a gene between adjacent target subsequences by measuring the results of recognition reactions on CDNA (or gDNA) mixtures. Of great importance, this method does not require the CDNA be inserted into a vector to create individual clones in a library. Creation of these libraries is time consuming, costly, and introduces bias into the process, as it requires the CDNA in the vector to be transformed into bacteria, the bacteria arrayed as clonal colonies, and finally the growth of the individual transformed colonies.

Three exemplary experimental methods are described herein for performing QEA: a preferred method utilizing a novel RE/ligase/amplification procedure; a PCR-based method; and a method utilizing a removal means, preferably biotin, for removal of unwanted DNA fragments. The preferred method generates precise, reproducible, noise free signatures for determining individual gene expression from DNA in mixtures or libraries and is uniquely adaptable to automation) since it does not require intermediate extractions or buffer exchanges. A computer implemented gene calling step uses the hit and length information measured in conjunction with a database of DNA sequences to determine which genes are present in the sample and the relative levels of expression. Signal intensities are used to determine relative amounts of sequences in the sample. Computer implemented design methods optimize the choice of the target subsequences.

A second specific embodiment of the invention, termed colony calling (“CC”), gathers only target subsequence presence information for all target subsequences for arrayed, individual single sequence clones in a library, with CDNA libraries being preferred. The target subsequences are carefully chosen according to computer implemented design methods of this invention to have a maximum information content and to be minimum in number. Preferably from 10-20 subsequences are sufficient to characterize the expressed CDNA in a tissue. In order to increase the specificity and reliability of hybridization to the typically short DNA subsequences, preferable recognition means are PNAs. Degenerate sets of longer DNA oligomers having a common, short, shared, target sequence can also be used as a recognition means. A computer implemented gene calling step uses the pattern of hits in conjunction with a database of DNA sequences to determine which genes are present in the sample and the relative levels of expression.

The embodiments of this invention preferably generate measurements that are precise, reproducible, and free of noise. Measurement noise in QEA is typically created by generation or amplification of unwanted DNA fragments, and special steps are preferably taken to avoid any such unwanted fragments. Measurement noise in colony calling is typically created by mis-hybridization of probes, or recognition means, to colonies. High stringency reaction conditions and DNA mimics with increased hybridization specificity may be used to minimize this noise. DNA mimics are polymers composed of subunits capable of specific, Watson-Crick-like hybridization with DNA. Also useful to minimize noise in colony calling are improved hybridization detection methods. Instead of the conventional detection methods based on probe labeling with fluorochromes, new methods are based on light scattering by small 100-200 μm particles that are aggregated upon probe hybridization (Stimson et al., 1995, “Real-time detection of DNA hybridization and melting on oligonucleotide arrays by using optical wave guides”, Proc. Natl. Acad. Sci. USA, 92:6379-6383). In this method, the hybridization surface forms one surface of a light pipe or optical wave guide, and the scattering induced by these aggregated particles causes light to leak from the light pipe. In this manner hybridization is revealed as an illuminated spot of leaking light on a dark background. This latter method makes hybridization detection more rapid by eliminating the need for a washing step between the hybridization and detection steps. Further by using variously sized and shaped particles with different light scattering properties, multiple probe hybridizations can be detected from one colony.

Further, the embodiments of the invention can be adapted to automation by eliminating non-automatable steps, such as extractions or buffer exchanges. The embodiments of the invention facilitate efficient analysis by permitting multiple recognition means to be tested in one reaction and by utilizing multiple, distinguishable labeling of the recognition means, so that signals may be simultaneously detected and measured. Preferably, for the QEA embodiments, this labeling is by multiple fluorochromes. For the CC embodiments, detection is preferably done by the light scattering methods with variously sized and shaped particles.

An increase in sensitivity as well as an increase in the number of resolvable fluorescent labels can be achieved by the use of fluorescent, energy transfer, or dye-labeled primers. Other detection methods, preferable when the genes being identified will be physically isolated from the gel for later sequencing or use as experimental probes, include the use of silver staining gels or of radioactive labeling. Since these methods do not allow for multiple samples to be run in a single lane, they are less preferable when high throughput is needed.

In biological research, rapid and economical assay for gene expression in tissue or other samples has numerous applications. Such applications include, but are not limited to, for example, in pathology examining tissue specific genetic response to disease, in embryology determining developmental changes in gene expression, in pharmacology assessing direct and indirect effects of drugs on gene expression. In these applications, this invention can be applied, e.g., to in vitro cell populations or cell lines, to in vivo animal models of disease or other processes, to human samples, to purified cell populations perhaps drawn from actual wild-type occurrences, and to tissue samples containing mixed cell populations. The cell or tissue sources can advantageously be a plant, a single celled animal, a multicellular animal, a bacterium, a virus, a fungus, or a yeast, etc. The animal can advantageously be laboratory animals used in research, such as mice engineered or bred to have certain genomes or disease conditions or tendencies. The in vitro cell populations or cell lines can be exposed to various exogenous factors to determine the effect of such factors on gene expression. Further, since an unknown signal pattern is indicative of an as yet unknown gene, this invention has important use for the discovery of new genes. In medical research, by way of further example, use of the methods of this invention allow correlating gene expression with the presence and progress of a disease and thereby provide new methods of diagnosis and new avenues of therapy which seek to directly alter gene expression.

This invention includes various embodiments and aspects, several of which are described below.

In a first embodiment, the invention provides a method for identifying, classifying, or quantifying one or more nucleic acids in a sample obtained from a microsome including a plurality of nucleic acids having different nucleotide sequences, the method including probing the sample with one or more recognition means, each recognition means recognizing a different target nucleotide subsequence or a different set of target nucleotide subsequences; generating one or more signals from the sample probed by the recognition means, each generated signal arising from a nucleic acid in the sample and including a representation of (i) the length between occurrences of target subsequences in the nucleic acid and (ii) the identities of the target subsequences in the nucleic acid or the identities of the sets of target subsequences among which is included the target subsequences in the nucleic acid; and searching a nucleotide sequence database to determine sequences that match or the absence of any sequences that match the one or more generated signals, the database including a plurality of known nucleotide sequences of nucleic acids that may be present in the sample, a sequence from the database matching a generated signal when the sequence from the database has both (i) the same length between occurrences of target subsequences as is represented by the generated signal and (ii) the same target subsequences as is represented by the generated signal, or target subsequences that are members of the same sets of target subsequences represented by the generated signal, whereby the one or more nucleic acids in the sample are identified, classified, or quantified.

This invention further provides in the first embodiment additional methods wherein each recognition means recognizes one target subsequence, and wherein a sequence from the database matches a generated signal when the sequence from the database has both the same length between occurrences of target subsequences as is represented by the generated signal and the same target subsequences as represented by the generated signal, or optionally wherein each recognition means recognizes a set of target subsequences, and wherein a sequence from the database matches a generated signal when the sequence from the database has both the same length between occurrences of target subsequences as is represented by the generated signal, and target subsequences that are members of the sets of target subsequences represented by the generated signal.

This invention further provides in the first embodiment additional methods further including dividing the sample of nucleic acids into a plurality of portions and performing the methods of this object individually on a plurality of the portions, wherein a different one or more recognition means are used with each portion.

This invention further provides in the first embodiment additional methods wherein the quantitative abundance of a nucleic acid including a particular nucleotide sequence in the sample is determined from the quantitative level of the one or more signals generated by the nucleic acid that are determined to match the particular nucleotide sequence.

This invention further provides in the first embodiment additional methods wherein the plurality of nucleic acids are DNA, and optionally wherein the DNA is cDNA, and optionally wherein the cDNA is prepared from a plant, an single celled animal, a multicellular animal, a bacterium, a virus, a fungus, or a yeast, and optionally wherein the cDNA is of total cellular RNA or total cellular poly(A) RNA.

This invention further provides in the first embodiment additional methods wherein the database comprises substantially all the known expressed sequences of the plant, single celled animal, multicellular animal, bacterium, or yeast.

This invention further provides in the first embodiment additional methods wherein the recognition means are one or more restriction endonucleases whose recognition sites are the target subsequences, and wherein the step of probing comprises digesting the sample with the one or more restriction endonucleases into fragments and ligating double stranded adapter DNA molecules to the fragments to produce ligated fragments, each the adapter DNA molecule including (i) a shorter stand having no 5′ terminal phosphates and consisting of a first and second portion, the first portion at the 5′ end of the shorter strand being complementary to the overhang produced by one of the restriction endonucleases and (ii) a longer strand having a 3′ end subsequence complementary to the second portion of the shorter strand; and wherein the step of generating further comprises melting the shorter strand from the ligated fragments, contacting the sample with a DNA polymerase, extending the ligated fragments by synthesis with the DNA polymerase to produce blunt-ended double stranded DNA fragments, and amplifying the blunt-ended fragments by a method including contacting the blunt-ended fragments with a DNA polymerase and primer oligodeoxynucleotides, the primer oligodeoxynucleotides including the longer adapter strand, and the contacting being at a temperature not greater than the melting temperature of the primer oligodeoxynucleotide from a strand of the blunt-ended fragments complementary to the primer oligodeoxynucleotide and not less than the melting temperature of the shorter strand of the adapter nucleic acid from the blunt-ended fragments.

This invention further provides in the first embodiment additional methods wherein the recognition means are one or more restriction endonucleases whose recognition sites are the target subsequences, and wherein the step of probing further comprises digesting the sample with the one or more restriction endonucleases.

This invention further provides in the first embodiment additional methods further including identifying a fragment of a nucleic acid in the sample which generates the one or more signals; and recovering the fragment, and optionally wherein the signals generated by the recovered fragment do not match a sequence in the nucleotide sequence database, and optionally further including using at least a hybridizable portion of the fragment as a hybridization probe to bind to a nucleic acid that can generate the fragment upon digestion by the one or more restriction endonucleases.

This invention further provides in the first embodiment additional methods wherein the step of generating further comprises after the digesting removing from the sample both nucleic acids which have not been digested and nucleic acid fragments resulting from digestion at only a single terminus of the fragments, and optionally wherein prior to digesting, the nucleic acids in the sample are each bound at one terminus to a biotin molecule or to a hapten molecule, and the removing is carried out by a method which comprises contacting the nucleic acids in the sample with streptavidin or avidin or with an anti-hapten antibody, respectively, affixed to a solid support.

This invention further provides in the first embodiment additional methods wherein the digesting with the one or more restriction endonucleases leaves single-stranded nucleotide overhangs on the digested ends.

This invention further provides in the first embodiment additional methods wherein the step of probing further comprises hybridizing double-stranded adapter nucleic acids with the digested sample fragments, each the adapter nucleic acid having an end complementary to the overhang generated by a particular one of the one or more restriction endonucleases, and ligating with a ligase a strand of the adapter nucleic acids to the 5′ end of a strand of the digested sample fragments to form ligated nucleic acid fragments.

This invention further provides in the first embodiment additional methods wherein the digesting with the one or more restriction endonucleases and the ligating are carried out in the same reaction medium, and optionally wherein the digesting and the ligating comprises incubating the reaction medium at a first temperature and then at a second temperature; in which the one or more restriction endonucleases are more active at the first temperature than the second temperature and the ligase is more active at the second temperature that the first temperature, or wherein the incubating at the first temperature and the incubating at the second temperature are performed repetitively.

This invention further provides in the first embodiment additional methods wherein the step of probing further comprises prior to the digesting removing terminal phosphates from DNA in the sample by incubation with an alkaline phosphatase, and optionally wherein the alkaline phosphatase is heat labile and is heat inactivated prior to the digesting.

This invention further provides in the first embodiment additional methods wherein the generating step comprises amplifying the ligated nucleic acid fragments, and optionally wherein the amplifying is carried out by use of a nucleic acid polymerase and primer nucleic acid strands, the primer nucleic acid strands being capable of priming nucleic acid synthesis by the polymerase, and optionally wherein the primer nucleic acid strands have a G+C content of between 40% and 60%.

This invention further provides in the first embodiment additional methods wherein each the adapter nucleic acid has a shorter strand and a longer strand, the longer strand being ligated to the digested sample fragments, and the generating step comprises prior to the amplifying step the melting of the shorter strand from the ligated fragments, contacting the ligated fragments with a DNA polymerase, extending the ligated fragments by synthesis with the DNA polymerase to produce blunt-ended double stranded DNA fragments, and wherein the primer nucleic acid strands comprise a hybridizable portion the sequence of the longer strands, or optionally comprise the sequence of the longer strands, each different primer nucleic acid strand priming amplification only of blunt ended double stranded DNA fragments that are produced after digestion by a particular restriction endonuclease.

This invention further provides in the first embodiment additional methods wherein each primer nucleic acid strand is specific for a particular restriction endonuclease, and further comprises at the 3′ end of and contiguous with the longer strand sequence the portion of the restriction endonuclease recognition site remaining on a nucleic acid fragment terminus after digestion by the restriction endonuclease, or optionally wherein each the primer specific for a particular restriction endonuclease further comprises at its 3′ end one or more nucleotides 3′ to and contiguous with the remaining portion of the restriction endonuclease recognition site, whereby the ligated nucleic acid fragment amplified is that including the remaining portion of the restriction endonuclease recognition site contiguous to the one or more additional nucleotides, and optionally such that the primers including a particular the one or more additional nucleotides can be distinguishably detected from the primers including a different the one or more additional nucleotides.

This invention further provides in the first embodiment additional methods wherein during the amplifying step the primer nucleic acid strands are annealed to the ligated nucleic acid fragments at a temperature that is less than the melting temperature of the primer nucleic acid strands from strands complementary to the primer nucleic acid strands but greater than the melting temperature of the shorter adapter strands from the blunt-ended fragments.

This invention further provides in the first embodiment additional methods wherein the recognition means are oligomers of nucleotides, nucleotide-mimics, or a combination of nucleotides and nucleotide-mimics, which are specifically hybridizable with the target subsequences, and optionally further provides additional methods wherein the step of generating comprises amplifying with a nucleic acid polymerase and with primers including the oligomers, whereby fragments of nucleic acids in the sample between hybridized oligomers are amplified.

This invention further provides in the first embodiment additional methods wherein the signals further comprise a representation of whether an additional target subsequence is present on the nucleic acid in the sample between the occurrences of target subsequences, and optionally wherein the additional target subsequence is recognized by a method comprising contacting nucleic acids in the sample with oligomers of nucleotides, nucleotide-mimics, or mixed nucleotides and nucleotide-mimics, which are hybridizable with the additional target subsequence.

This invention further provides in the first embodiment additional methods wherein the step of generating comprises suppressing the signals when an additional target subsequence is present on the nucleic acid in the sample between the occurrences of target subsequences, and optionally wherein, when the step of generating comprises amplifying nucleic acids in the sample, the additional target subsequence is recognized by a method comprising contacting nucleic acids in the sample with (a) oligomers of nucleotides, nucleotide-mimics, or mixed nucleotides and nucleotide-mimics, which hybridize with the additional target subsequence and disrupt the amplifying step; or (b) restriction endonucleases which have the additional target subsequence as a recognition site and digest the nucleic acids in the sample at the recognition site.

This invention further provides in the first embodiment additional methods wherein the step of generating further comprises separating nucleic acid fragments by length, and optionally wherein the step of generating further comprises detecting the separated nucleic acid fragments, and optionally wherein the detecting is carried out by a method comprising staining the fragments with silver, labeling the fragments with a DNA intercalating dye, or detecting light emission from a fluorochrome label on the fragments.

This invention further provides in the first embodiment additional methods wherein the representation of the length between occurrences of target subsequences is the length of fragments determined by the separating and detecting steps.

This invention further provides in the first embodiment additional methods wherein the separating is carried out by use of liquid chromatography, mass spectrometry, or electrophoresis, and optionally wherein the electrophoresis is carried out in a slab gel or capillary configuration using a denaturing or non-denaturing medium.

This invention further provides in the first embodiment additional methods wherein a predetermined one or more nucleotide sequences in the database are of interest, and wherein the target subsequences are such that the sequences of interest generate at least one signal that is not generated by any other sequence likely to be present in the sample, and optionally wherein the nucleotide sequences of interest are a majority of sequences in the database.

This invention further provides in the first embodiment additional methods wherein the target subsequences have a probability of occurrence in the nucleotide sequences in the database of from approximately 0.01 to approximately 0.30.

This invention further provides in the first embodiment additional methods wherein the target subsequences are such that the majority of sequences in the database contain on average a sufficient number of occurrences of target subsequences in order to on average generate a signal that is not generated by any other nucleotide sequence in the database, and optionally wherein the number of pairs of target subsequences present on average in the majority of sequences in the database is no less than 3, and wherein the average number of signals generated from the sequences in the database is such that the average difference between lengths represented by the generated signals is greater than or equal to 1 base pair.

This invention further provides in the first embodiment additional methods wherein the target subsequences have a probability of occurrence, p, approximately given by the solution of [(R(R+1)p²]/2=A, wherein N=the number of different nucleotide sequences in the database; L=the average length of the different nucleotide sequences in the database; R=the number of recognition means; A=the number of pairs of target subsequences present on average in the different nucleotide sequences in the database; and B=the average difference between lengths represented by the signals generated from the nucleic acids in the sample, and optionally wherein A is greater than or equal to 3 and wherein B is greater than or equal to 1.

This invention further provides in the first embodiment additional methods wherein the target subsequences are selected according to the further steps comprising determining a pattern of signals that can be generated and the sequences capable of generating each such signal by simulating the steps of probing and generating applied to each sequences in the database of nucleotide sequences; ascertaining the value of the determined pattern according to an information measure; and choosing the target subsequences in order to generate a new pattern that optimizes the information measure, and optionally wherein the choosing step selects target subsequences which comprise the recognition sites of the one or more restriction endonucleases, and optionally wherein the choosing step selects target subsequences which comprise the recognition sites of the one or more restriction endonucleases contiguous with one or more additional nucleotides.

This invention further provides in the first embodiment additional methods wherein a predetermined one or more of the nucleotide sequences present in the database of nucleotide sequences are of interest, and the information measure optimized is the number of such the sequences of interest which generate at least one signal that is not generated by any other nucleotide sequence present in the database, and optionally wherein the nucleotide sequences of interest are a majority of the nucleotide sequences present in the database.

This invention further provides in the first embodiment additional methods wherein the choosing step is by exhaustive search of all combinations of target subsequences of length less than approximately 10, or wherein the step of choosing target subsequences is by a method comprising simulated annealing.

This invention further provides in the first embodiment additional methods wherein the step of searching further comprises determining a pattern of signals that can be generated and the sequences capable of generating each such signal by simulating the steps of probing and generating applied to each sequence in the database of nucleotide sequences; and finding the one or more nucleotide sequences in the database that are able to generate the one or more generated signals by finding in the pattern those signals that comprise a representation of the (i) the same lengths between occurrences of target subsequences as is represented by the generated signal and (ii) the same target subsequences as is represented by the generated signal, or target subsequences that are members of the same sets of target subsequences represented by the generated signal.

This invention further provides in the first embodiment additional methods wherein the step of determining further comprises searching for occurrences of the target subsequences or sets of target subsequences in nucleotide sequences in the database of nucleotide sequences; finding the lengths between occurrences of the target subsequences or sets of target subsequences in the nucleotide sequences of the database; and forming the pattern of signals that can be generated from the sequences of the database in which the target subsequences were found to occur.

This invention further provides in the first embodiment additional methods wherein the restriction endonucleases generate 5′ overhangs at the terminus of digested fragments and wherein each double stranded adapter nucleic acid comprises a shorter nucleic acid strand consisting of a first and second contiguous portion, the first portion being a 5′ end subsequence complementary to the overhang produced by one of the restriction endonucleases; and a longer nucleic acid strand having a 3′ end subsequence complementary to the second portion of the shorter strand.

This invention further provides in the first embodiment additional methods wherein the shorter strand has a melting temperature from a complementary strand of less than approximately 68.degree. C., and has no terminal phosphate, and optionally wherein the shorter strand is approximately 12 nucleotides long.

This invention further provides in the first embodiment additional methods wherein the longer strand has a melting temperature from a complementary strand of greater than approximately 68.degree. C., is not complementary to any nucleotide sequence in the database, and has no terminal phosphate, and optionally wherein the ligated nucleic acid fragments do not contain a recognition site for any of the restriction endonucleases, and optionally wherein the longer strand is approximately 24 nucleotides long and has a G+C content between 40% and 60%.

This invention further provides in the first embodiment additional methods wherein the one or more restriction endonucleases are heat inactivated before the ligating.

This invention further provides in the first embodiment additional methods wherein the restriction endonucleases generate 3′ overhangs at the terminus of the digested fragments and wherein each double stranded adapter nucleic acid comprises a longer nucleic acid strand consisting of a first and second contiguous portion, the first portion being a 3′ end subsequence complementary to the overhang produced by one of the restriction endonucleases; and a shorter nucleic acid strand complementary to the 3′ end of the second portion of the longer nucleic acid stand.

This invention further provides in the first embodiment additional methods wherein the shorter strand has a melting temperature from the longer strand of less than approximately 68.degree. C., and has no terminal phosphates, and optionally wherein the shorter strand is 12 base pairs long.

This invention further provides in the first embodiment additional methods wherein the longer strand has a melting temperature from a complementary strand of greater than approximately 68.degree. C., is not complementary to any nucleotide sequence in the database, has no terminal phosphate, and wherein the ligated nucleic acid fragments do not contain a recognition site for any of the restriction endonucleases, and optionally wherein the longer strand is 24 base pairs long and has a G+C content between 40% and 60%.

In a second embodiment, the invention provides a method for identifying or classifying a nucleic acid isolated from a microsome or derived from microsomal RNA, comprising probing the nucleic acid with a plurality of recognition means, each recognition means recognizing a target nucleotide subsequence or a set of target nucleotide subsequences, in order to generate a set of signals, each signal representing whether the target subsequence or one of the set of target subsequences is present or absent in the nucleic acid; and searching a nucleotide sequence database, the database comprising a plurality of known nucleotide sequences of nucleic acids that may be present in the sample, for sequences matching the generated set of signals, a sequence from the database matching a set of signals when the sequence from the database (i) comprises the same target subsequences as are represented as present, or comprises target subsequences that are members of the sets of target subsequences represented as present by the generated sets of signals and (ii) does not comprise the target subsequences represented as absent or that are members of the sets of target subsequences represented as absent by the generated sets of signals, whereby the nucleic acid is identified or classified, and optionally wherein the set of signals are represented by a hash code which is a binary number.

This invention further provides in the second embodiment additional methods wherein the step of probing generates quantitative signals of the numbers of occurrences of the target subsequences or of members of the set of target subsequences in the nucleic acid, and optionally wherein a sequence matches the generated set of signals when the sequence from the database comprises the same target subsequences with the same number of occurrences in the sequence as in the quantitative signals and does not comprise the target subsequences represented as absent or target subsequences within the sets of target subsequences represented as absent.

This invention further provides in the second embodiment additional methods wherein the plurality of nucleic acids are DNA.

This invention further provides in the second embodiment additional methods wherein the recognition means are detectably labeled oligomers of nucleotides, nucleotide-mimics, or combinations of nucleotides and nucleotide-mimics, and the step of probing comprises hybridizing the nucleic acid with the oligomers, and optionally wherein the detectably labeled oligomers are detected by a method comprising detecting light emission from a fluorochrome label on the oligomers or arranging the labeled oligomers to cause light to scatter from a light pipe and detecting the scattering, and optionally wherein the recognition means are oligomers of peptide-nucleic acids, and optionally wherein the recognition means are DNA oligomers, DNA oligomers comprising universal nucleotides, or sets of partially degenerate DNA oligomers.

This invention further provides in the second embodiment additional methods wherein the step of searching further comprises determining a pattern of sets of signals of the presence or absence of the target subsequences or the sets of target subsequences that can be generated and the sequences capable of generating each set of signals in the pattern by simulating the step of probing as applied to each sequence in the database of nucleotide sequences; and finding one or more nucleotide sequences that arc capable of generating the generated set of signals by finding in the pattern those sets that match the generated set, where a set of signals from the pattern matches a generated set of signals when the set from the pattern (i) represents as present the same target subsequences as are represented as present or target subsequences that are members of the sets of target subsequences represented as present by the generated sets of signals and (ii) represents as absent the target subsequences represented as absent or that are members of the sets of target subsequences represented as absent by the generated sets of signals.

This invention further provides in the second embodiment additional methods wherein the target subsequences are selected according to the further steps comprising determining (i) a pattern of sets of signals representing the presence or absence of the target subsequences or of the sets of target subsequences that can be generated, and (ii) the sequences capable of generating each set of signals in the pattern by simulating the step of probing as applied to each sequence in the database of nucleotide sequences; ascertaining the value of the pattern generated according to an information measure; and choosing the target subsequences in order to generate a new pattern that optimizes the information measure.

This invention further provides in the second embodiment additional methods wherein the information measure is the number of sets of signals in the pattern which are capable of being generated by one or more sequences in the database, or optionally wherein the information measure is the number of sets of signals in the pattern which are capable of being generated by only one sequence in the database.

This invention further provides in the second embodiment additional methods wherein the choosing step is by a method comprising exhaustive search of all combination of target subsequences of length less than approximately 10, or optionally wherein the choosing step is by a method comprising simulated annealing.

This invention further provides in the second embodiment additional methods wherein the step of determining by simulating further comprises searching for the presence or absence of the target subsequences or sets of target subsequences in each nucleotide sequence in the database of nucleotide sequences; and forming the pattern of sets of signals that can be generated from the sequences in the database, and optionally where the step of searching is carried out by a string search, and optionally wherein the step of searching comprises counting the number of occurrences of the target subsequences in each nucleotide sequence.

This invention further provides in the second embodiment additional methods wherein the target subsequences have a probability of occurrence in a nucleotide sequence in the database of nucleotide sequences of from 0.01 to 0.6, or optionally wherein the target subsequences are such that the presence of one target subsequence in a nucleotide sequence in the database of nucleotide sequences is substantially independent of the presence of any other target subsequence in the nucleotide sequence, or optionally wherein fewer than approximately 50 target subsequences are selected.

In a third embodiment, the invention provides a method for identifying, classifying, or quantifying DNA molecules in a sample of DNA molecules derived from microsomal RNA having a plurality of different nucleotide sequences, the method comprising the steps of digesting the sample with one or more restriction endonucleases, each the restriction endonuclease recognizing a subsequence recognition site and digesting DNA at the recognition site to produce fragments with 5′ overhangs; contacting the fragments with shorter and longer oligodeoxynucleotides, each the shorter oligodeoxynucleotide hybridizable with a the 5′ overhang and having no terminal phosphates, each the longer oligodeoxynucleotide hybridizable with a the shorter oligodeoxynucleotide; ligating the longer oligodeoxynucleotides to the 5′ overhangs on the DNA fragments to produce ligated DNA fragments; extending the ligated DNA fragments by synthesis with a DNA polymerase to produce blunt-ended double stranded DNA fragments; amplifying the blunt-ended double stranded DNA fragments by a method comprising contacting the DNA fragments with a DNA polymerase and primer oligodeoxynucleotides, each the primer oligodeoxynucleotide having a sequence comprising that of one of the longer oligodeoxynucleotides; determining the length of the amplified DNA fragments; and searching a DNA sequence database, the database comprising a plurality of known DNA sequences that may be present in the sample, for sequences matching one or more of the fragments of determined length, a sequence from the database matching a fragment of determined length when the sequence from the database comprises recognition sites of the one or more restriction endonucleases spaced apart by the determined length, whereby DNA molecules in the sample are identified, classified, or quantified.

This invention further provides in the third embodiment additional methods wherein the sequence of each primer oligodeoxynucleotide further comprises 3′ to and contiguous with the sequence of the longer oligodeoxynucleotide the portion of the recognition site of the one or more restriction endonucleases remaining on a DNA fragment terminus after digestion, the remaining portion being 5′ to and contiguous with one or more additional nucleotides, and wherein a sequence from the database matches a fragment of determined length when the sequence from the database comprises subsequences that are the recognition sites of the one or more restriction endonucleases contiguous with the one or more additional nucleotides and when the subsequences are spaced apart by the determined length.

This invention further provides in the third embodiment additional methods wherein the determining step further comprises detecting the amplified DNA fragments by a method comprising staining the fragments with silver. This invention further provides in the third embodiment additional methods wherein the oligodeoxynucleotide primers are detectably labeled, wherein the determining step further comprises detection of the detectable labels, and wherein a sequence from the database matches a fragment of determined length when the sequence from the database comprises recognition sites of the one or more restriction endonucleases, the recognition sites being identified by the detectable labels of the oligodeoxynucleotide primers, the recognition sites being spaced apart by the determined length, and optionally wherein the determining step further comprises detecting the amplified DNA fragments by a method comprising labeling the fragments with a DNA intercalating dye or detecting light emission from a fluorochrome label on the fragments.

This invention further provides in the third embodiment additional steps further comprising, prior to the determining step, the step of hybridizing the amplified DNA fragments with a detectably labeled oligodeoxynucleotide complementary to a subsequence, the subsequence differing from the recognition sites of the one or more restriction endonucleases, wherein the determining step further comprises detecting the detectable label of the oligodeoxynucleotide, and wherein a sequence from the database matches a fragment of determined length when the sequence from the database further comprises the subsequence between the recognition sites of the one or more restriction endonucleases.

This invention further provides in the third embodiment additional methods wherein the one or more restriction endonucleases are pairs of restriction endonucleases, the pairs being selected from the group consisting of Acc56I and HindIII, Acc65I and NgoMI, BamHI and EcoRI, BgIII and HindIII, BgIII and NgoMI, BsiWI and BspHI, BspHI and BstYI, BspHI and NgoMI, BsrGI and EcoRI, EagI and EcoRI, EagI and HindIII, EagI and NcoI, HindIII and NgoMI, NgoMI and NheI, NgoMI and SpeI, BgIII and BspHI, Bsp120I and NcoI, BssHII and NgoMI, EcoRI and HindIII, and NgoMI and XbaI, or wherein the step of ligating is performed with T4 DNA ligase.

This invention further provides in the third embodiment additional methods wherein the steps of digesting, contacting, and ligating are performed simultaneously in the same reaction vessel, or optionally wherein the steps of digesting, contacting, ligating, extending, and amplifying are performed in the same reaction vessel.

This invention further provides in the third embodiment additional methods wherein the step of determining the length is performed by electrophoresis.

This invention further provides in the third embodiment additional methods wherein the step of searching the DNA database further comprises determining a pattern of fragments that can be generated and for each fragment in the pattern those sequences in the DNA database that are capable of generating the fragment by simulating the steps of digesting with the one or more restriction endonucleases, contacting, ligating, extending, amplifying, and determining applied to each sequence in the DNA database; and finding the sequences that are capable of generating the one or more fragments of determined length by finding in the pattern one or more fragments that have the same length and recognition sites as the one or more fragments of determined length.

This invention further provides in the third embodiment additional methods wherein the steps of digesting and ligating go substantially to completion.

This invention further provides in the third embodiment additional methods wherein the DNA sample is cDNA prepared from mRNA, and optionally wherein the DNA is of RNA from a tissue or a cell type derived from a plant, a single celled animal, a multicellular animal, a bacterium, a virus, a fungus, a yeast, or a mammal, and optionally wherein the mammal is a human, and optionally wherein the mammal is a human having or suspected of having a diseased condition, and optionally wherein the diseased condition is a malignancy.

In a fourth embodiment, this invention provides additional methods for identifying, classifying, or quantifying DNA molecules in a sample of DNA molecules derived from microsomal RNA with a plurality of nucleotide sequences, the method comprising the steps of digesting the sample with one or more restriction endonucleases, each the restriction endonuclease recognizing a subsequence recognition site and digesting DNA to produce fragments with 3′ overhangs; contacting the fragments with shorter and longer oligodeoxynucleotides, each the longer oligodeoxynucleotide consisting of a first and second contiguous portion, the first portion being a 3′ end subsequence complementary to the overhang produced by one of the restriction endonucleases, each the shorter oligodeoxynucleotide complementary to the 3′ end of the second portion of the longer oligodeoxynucleotide stand; ligating the longer oligodeoxynucleotide to the DNA fragments to produce a ligated fragment; extending the ligated DNA fragments by synthesis with a DNA polymerase to form blunt-ended double stranded DNA fragments; amplifying the double stranded DNA fragments by use of a DNA polymerase and primer oligodeoxynucleotides to produce amplified DNA fragments, each the primer oligodeoxynucleotide having a sequence comprising that of a longer oligodeoxynucleotide; determining the length of the amplified DNA fragments; and searching a DNA sequence database, the database comprising a plurality of known DNA sequences that may be present in the sample, for sequences matching one or more of the fragments of determined length, a sequence from the database matching a fragment of determined length when the sequence from the database comprises recognition sites of the one or more restriction endonucleases spaced apart by the determined length, whereby DNA sequences in the sample are identified, classified, or quantified.

In a fifth embodiment, this invention provides additional methods of detecting one or more differentially expressed genes in an in vitro cell exposed to an exogenous factor relative to an in vitro cell not exposed to the exogenous factor comprising performing the methods the first embodiment of this invention wherein the plurality of nucleic acids comprises cDNA of RNA isolated from a microsome of the in vitro cell exposed to the exogenous factor; performing the methods of the first embodiment of this invention wherein the plurality of nucleic acids comprises cDNA of RNA of the in vitro cell not exposed to the exogenous factor; and comparing the identified, classified, or quantified cDNA of the in vitro cell exposed to the exogenous factor with the identified, classified, or quantified cDNA of the in vitro cell not exposed to the exogenous factor, whereby differentially expressed genes are identified, classified, or quantified.

In a sixth embodiment, this invention provides additional methods of detecting one or more differentially expressed genes in a diseased tissue relative to a tissue not having the disease comprising performing the methods of the first embodiment of this invention wherein the plurality of nucleic acids comprises cDNA of RNA isolated from a microsome of the diseased tissue such that one or more cDNA molecules are identified, classified, and/or quantified; performing the methods of the first embodiment of this invention wherein the plurality of nucleic acids comprises cDNA of RNA of the tissue not having the disease such that one or more cDNA molecules are identified, classified, and/or quantified; and comparing the identified, classified, and/or quantified cDNA molecules of the diseased tissue with the identified, classified, and/or quantified cDNA molecules of the tissue not having the disease, whereby differentially expressed cDNA molecules are detected.

This invention further provides in the sixth embodiment additional methods wherein the step of comparing further comprises finding CDNA molecules which are reproducibly expressed in the diseased tissue or in the tissue not having the disease and further finding which of the reproducibly expressed CDNA molecules have significant differences in expression between the tissue having the disease and the tissue not having the disease, and optionally wherein the finding cDNA molecules which are reproducibly expressed and the significant differences in expression of the CDNA molecules in the diseased tissue and in the tissue not having the disease are determined by a method comprising applying statistical measures, and optionally wherein the statistical measures comprise determining reproducible expression if the standard deviation of the level of quantified expression of a cDNA molecule in the diseased tissue or the tissue not having the disease is less than the average level of quantified expression of the CDNA molecule in the diseased tissue or the tissue not having the disease, respectively, and wherein a cDNA molecule has significant differences in expression if the sum of the standard deviation of the level of quantified expression of the cDNA molecule in the diseased tissue plus the standard deviation of the level of quantified expression of the cDNA molecule in the tissue not having the disease is less than the absolute value of the difference of the level of quantified expression of the cDNA molecule in the diseased tissue minus the level of quantified expression of the cDNA molecule in the tissue not having the disease.

This invention further provides in the sixth embodiment additional methods wherein the diseased tissue and the tissue not having the disease are from one or more mammals, and optionally wherein the disease is a malignancy, and optionally wherein the disease is a malignancy selected from the group consisting of prostrate cancer, breast cancer, colon cancer, lung cancer, skin cancer, lymphoma, and leukemia.

This invention further provides in the sixth embodiment additional methods wherein the disease is a malignancy and the tissue not having the disease has a premalignant character.

In a seventh embodiment, this invention provides methods of staging or grading a disease in a human individual comprising performing the methods of the first embodiment of this invention in which the plurality of nucleic acids comprises cDNA of RNA isolated from a microsome prepared from a tissue from the human individual, the tissue having or suspected of having the disease, whereby one or more the CDNA molecules are identified, classified, and/or quantified; and comparing the one or more identified, classified, and/or quantified CDNA molecules in the tissue to the one or more identified, classified, and/or quantified CDNA molecules expected at a particular stage or grade of the disease.

In an eighth embodiment, this invention provides additional methods for predicting a human patient's response to therapy for a disease, comprising performing the methods of the first embodiment of this invention in which the plurality of nucleic acids comprises cDNA of RNA isolated from a microsome prepared from a tissue from the human patient, the tissue having or suspected of having the disease, whereby one or more CDNA molecules in the sample are identified, classified, and/or quantified; and ascertaining if the one or more CDNA molecules thereby identified, classified, and/or quantified correlates with a poor or a favorable response to one or more therapies, and optionally which further comprises selecting one or more therapies for the patient for which the identified, classified, and/or quantified CDNA molecules correlates with a favorable response.

In a ninth embodiment, this invention provides additional methods for evaluating the efficacy of a therapy in a mammal having a disease, the method comprising performing the methods of the first embodiment of this invention wherein the plurality of nucleic acids comprises cDNA of RNA isolated from a microsome of the mammal prior to a therapy; performing the method of the first embodiment of this invention wherein the plurality of nucleic acids comprises cDNA of RNA of the mammal subsequent to the therapy; comparing one or more identified, classified, and/or quantified cDNA molecules in the mammal prior to the therapy with one or more identified, classified, and/or quantified cDNA molecules of the mammal subsequent to therapy; and determining whether the response to therapy is favorable or unfavorable according to whether any differences in the one or more identified, classified, and/or quantified cDNA molecules after therapy are correlated with regression or progression, respectively, of the disease, and optionally wherein the mammal is a human.

The invention will be further illustrated in the following non-limiting examples. In Examples 1-2, expression patterns were compared between human ostcosarcoma MG-63 cells exposed to IL-1α and control cells not subjected to the growth factor. This experimental system was chosen for the following reasons: (a) MG-63 is a human osteosarcoma cell line, which can be differentiated into osteoblast-like cells or adipocytes by various treatments; (b) in vivo, osteoblast cells may produce and secrete factors that affect differentiation of hematopoietic precursors; (c) IL-1α is a pro-inflammatory cytokine known to exert biological effects on osteoblast cells; and (d) osteoblasts may participate in inflammatory events leading to the loss of bone mass. Thus, the response of MG-63 cells to IL-1α can reveal mechanisms by which osteoblasts recruit lymphocytes, promote inflammation, and regulate hematopoiesis, some of which might be controlled by translation up- or down-regulation. In Example 3, actively translated mRNAs encoding secreted or membrane-associated proteins were enriched from frozen tissue and cultured cells by isolating microsomes using sucrose gradient fractionation and SeqCalling™ technology.

### EXAMPLE 1

### General Materials and Methods

Cell Culture

Human osteosarcoma MG-63 cells were maintained in MEM containing 10% fetal bovine serum at 37° C. and 5% CO₂ with humidity. 3×10⁶ cells/T175 flask MG63 cells were serum starved in MEM media containing 0.1% FBS for 24 hours and then treated with 10 ng/ml IL-1α for 6 hours. Rabbit anti-CAML polyclonal antibody was a kind gift from Dr. Richard J. Brani (Department of Pediatrics, Immunology, Mayo Clinic, Rochester, Minn.). Mouse anti-β-actin monoclonal antibody was purchased from Santa Cruz Biotech (Santa Cruz, Calif.). Cycloheximide was purchased from ICN.

Polyribosome Analysis

For preparation of cytoplasmic extracts, cells from three 175 cm² tissue culture plates (30%) confluent were treated with cycloheximide (100 μg/ml; ICN) for 5 min. at 37° C., washed with ice cold PBS containing cycloheximide (100 μg/ml), and harvested by trypsinization (Johannes et al., PNAS 96:13118-13123, 1999). Cells and homogenates were also snap frozen in liquid nitrogen after cycloheximide treatment and harvesting. The fresh cells were pelleted by centrifugation, swollen for 2 min. in 375 μl of low salt buffer (LSB; 20 mM Tris pH 7.5, 10 mM NaCl, and 3 mM MgCl₂) containing I mM dithiothreitol and 50 units of recombinant RNasin (Promega), and lysed by addition of 125 μl of lysis buffer [1×LSB/0.2 M sucrose/1.2% Triton N-100 (Sigma)] followed by vortexing. The nuclei were pelleted by centrifugation in a microcentriflige at 13,000 rpm for 2 min. The supernatant (cytoplasmic extract) was transferred to a new 1.5 ml tube on ice. Cytoplasmic extracts were carefully layered over 0.5-1.5 M linear sucrose gradients (in LSB) and centrifuged at 45,000 rpm in a Beckman SW40 rotor for 90 min. at 4° C. Gradients were fractionated using a pipette, and then absorbance at 260 nm was measured from each fraction by UV spectrometry.

CDNA Sytillesis

The polysomal fractions from each sample were pooled together, and the RNAs from each sample were isolated using Trizol Reagent (GIBCO-BRL) and reverse transcribed to cDNA using oligo-dT primer and SuperScript II reverse transcriptase (GIBCO-BRL) using CuraGen's standard operating procedure for CDNA synthesis. (See, e.g., Pat. No. 5,871,697).

Gene Expression Analysis

QEA and gene expression analysis was performed essentially as previously outlined (Shimkets et al., Nature Biotech. 17:798-803, 1999). In brief, an individual QEA reaction consists of cDNA template, two restriction enzymes, a ligase, a thermostable DNA polymerase, and all other components necessary for the activity of each enzyme. QEA produces double stranded fluorescently labeled DNA. The labeled DNA is resolved by polyacrylamide gel electrophoresis and detected by a high resolution charge coupled device (CCD) cameras. The size of the QEA products are tracked in CuraGen Corporation's database and accessed via GeneScape™.

Western Immunoblot Analysis

MG-63 cells were harvested and processed as described (Sheikh et al., Oncogene 18: 6121-6128, 1999). Equal amounts of protein (100 jig) from each cells were resolved by SDS/PAGE on 12.5% gels by the method of Laemmli (Laemmli, Nature 227: 680-685, 1970). Proteins were probed with rabbit anti-CAML polyclonal antibody (1:4000 dilution), mouse anti β-actin monoclonal antibody (1:5000 dilution) followed by incubation with a horseradish peroxidase-conjugated secondary antibody (Bio-Rad). Proteins were visualized with a chemiluminescence detection system using the Super Signal substrate (Pierce).

### EXAMPLE 2

### . Identification of Gene Transcripts Present in Different Levels in Polysomal mRNA from IL-1α0 Treated MG-63 Cells

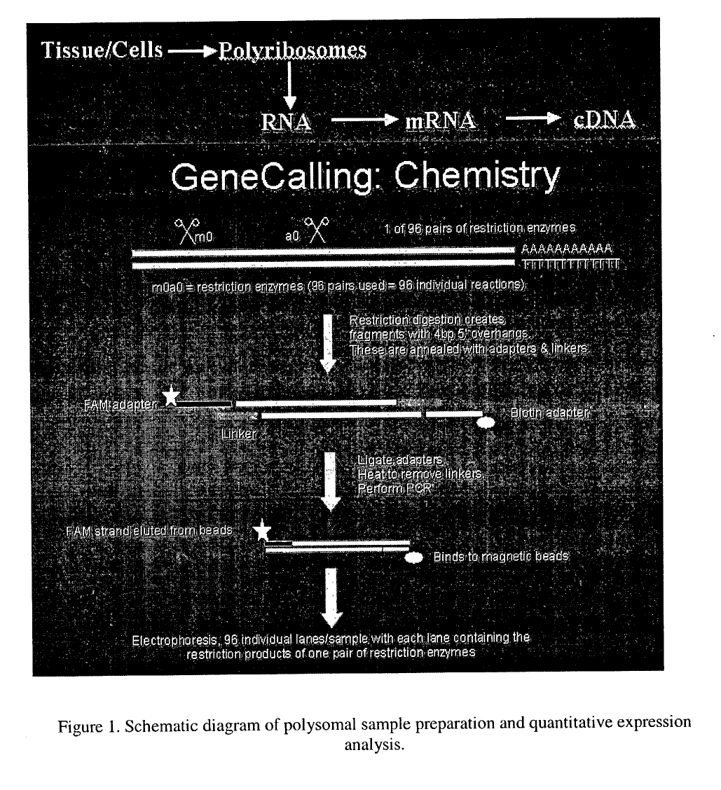

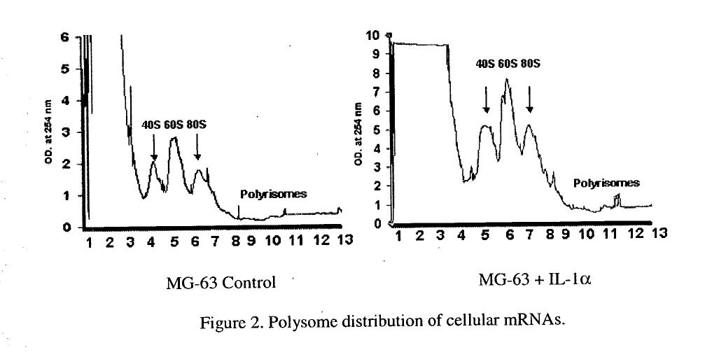

Gene expression from polysomal isolated mRNAs in serum starved MG-63 cells and MG-63 cells induced with inflammation cytokine IL-1α was analyzed, as is shown in FIG. 1. Polysomal mRNA was isolated from total cell mRNA by sucrose density sedimentation centrifugation on 0.5M-1.5M sucrose gradients. FIG. 2 shows the optical density (OD) profile of sucrose gradients loaded with cell extracts from untreated and IL-1α treated MG-63 cells. In each gradient the top fractions with high OD values represent ribosomal RNAs associated with the 40S, 60S , 80S subunits, along with free mRNAs. Sample fractions with lower ODs contain the polysomal fractions with actively translated mRNAs. For expression analysis, fractions 8 to 13 containing polysomes were pooled, the mRNA isolated and converted to cDNA for expression analysis. In addition, polysomes were isolated from snap frozen cells and homogenates and the polysome gene expression analysis results are consistent with the freshly isolated sample.

The cDNA was analyzed using the gene expression analysis technology essentially as described in Shimkets et al., Nature Biotech. 17:798-803, 1999. To achieve appropriate gene coverage typically 50-100 different restriction enzyme pairs were used per study. The amplified sample was analyzed by capillary gel electrophoresis, and each cDNA species was represented by one or multiple fragments of precisely defined size. The relative abundance of each fragment, and thereby the mRNA it was derived from, was determined. Gene identity was assigned to fragments representing genes previously known. In addition, this analysis platform allows the discovery of hitherto unknown gene products through the isolation and characterization of novel fragments.

Expression analysis by gene expression analysis of IL-1α-treated vs. untreated control samples yielded a total of 1709 differences for polysomal analysis using a total of 53 restriction enzyme pairs, and 1581 differences for the total mRNA samples using 86 restriction enzyme pairs. For the polysomal samples 12.5% of all monitored genes were differentially expressed (cut-off 2-fold) whereas for total mRNA the difference was smaller at 2.5%. The proportionally higher number of differentially expressed mRNAs in the polysomal pool presumably reflects the exclusion of non-translating mRNAs from this subpopulation. About 54% of the genes were transcriptionally regulated. Among them, 35% of the genes were differentially expressed in both total and polysomal mRNA and 19% are only differentially expressed in total mRNA gene expression analysis. These data reflect the complexity of the gene expression regulation during IL-1α treatment. Furthermore, the data demonstrate that it is absolutely critical to monitor gene expression at different levels of regulation.

Data from the two gene expression analysis analyses (total cellular mRNA and the polysomal mRNA) were compared. A set of genes, of which some are listed in Table 1, were identified as regulated at the transcriptional level. This demonstrates that genes that are transcriptionally induced with IL-1α were also translated to the same extent. Most of the listed genes were also confirmed with oligo poisoning, a method in which an antisense oligo binds to a corresponding target CDNA and eliminated from QEA fragment (Shimkets et al, Nature Biotech. 17:798-803, 1999).

TABLE 1
Genes potentially regulated at the transcriptional level.

| Gene Id | | | | | |
|---|---|---|---|---|---|
| gbh_m37719 | 100 | | 100 | [symbol] | Human monocyte chemotactic |
| | | | | | protein gene, complete cds. |
| uehsf_12961_0 | 100 | | 90 | [symbol] | yo61a11.rl Homo sapiens |
| | | | | | c DNA, 5″ end |
| gbh_m26383 | 14 | [symbol] | 60 | [symbol] | Human monocyte-derived |
| | | | | | neutrophil-activating protein |
| | | | | | (MONAP) |
| gbh_m92357 | 21 | | 36 | [symbol] | Homo sapiens tumor necrosis |
| | | | | | factor alpha-induced protein 2 |
| uehsf_40031_0 | 25 | | 20 | [symbol] | Human guanylate binding |
| | | | | | protien isoform I (GBP-2) |
| | | | | | mRNA, complete cds |
| gbh_af038963 | 11 | | 32 | | Homo sapiens RNA helicase |
| | | | | | RIG-I |
| gbh_m55542 | 25 | | 7 | [symbol] | Human guanylate binding |
| | | | | | protein isoform I mRNA, |
| | | | | | complete |
| gbh_m37435 | 16 | | 14 | [symbol] | Human macrophage-specific |
| | | | | | colony-stimulating factor |
| | | | | | (CSF-1) |
| gbh_m24594 | 20 | | 9 | [symbol] | Human interferon-induced |
| | | | | | 56 kD protien |
| gbh_149432 | 20 | | 9 | [symbol] | Homo sapiens TNFR2-TRAF |
| | | | | | signalling complex protein |
| | | | | | mRNA, complete |
| gbh_x57522 | 15 | | 11 | [symbol] | H. sapiens RING4 c DNA. |
| gbh_m30817 | 8 | | 15 | [symbol] | Human interferon-regulated |
| | | | | | resistance GTP-binding |
| | | | | | protein Mx A (ak |
| gbh_u56102 | 19 | [symbol] | 4 | | Human adhesion molecule |
| | | | | | DNAM-1 mRNA, complete |
| | | | | | cds. |
| gbh_121204 | 15 | | 8 | [symbol] | Homo sapiens antigen peptide |
| | | | | | transporter 1 |
| Gbh_u96922 | 8 | | 13 | [symbol] | Homo sapiens inositol poly- |
| | | | | | phosphate 4-phosphatase type |
| | | | | | II-alpha |
| gbh_105072 | 8 | | 12 | [symbol] | Homo sapiens interferon |
| | | | | | regulatory factor 1 |
| gbh_aj225089 | 14 | | 4 | [symbol] | Homo sapiens 59 kDa 2′-5′ |
| | | | | | oligoadenylate synthetase-like |
| | | | | | protein |
| gbh_u18420 | 14 | | 3 | [symbol] | Human ras-related small GTP |
| | | | | | binding protein Rab5 |
| | | | | | (rab5) mRNA. |
| gbh_m97936 | 8 | | 7 | [symbol] | Human transcription factor |
| | | | | | ISGF-3 mRNA sequence. |

The genes listed in Table 2 (part of the listed genes that were confirmed by poisoning) showed significant induction by IL-1α based upon steady-state total mRNA gene expression analysis. However, they showed no significant difference in mRNA levels obtained by polysome isolation. The results indicate that for certain genes, even though they were differentially expressed at the transcriptional level, differential expression was not reflected at translational level during the treatment time. It might be that cells are set a stage for a set of genes for later event corresponding to the early response genes at that time of treatment.

TABLE 2
Transcriptionally upregulated genes involved in cell signaling.

| Gene Id | | | |
|---|---|---|---|
| uehsf_1706_1 | −2 | 100 | yf50109 s1 Homo sapiens cDNA 3″ end |
| | | | SIM ATPase. Na+/K+ |
| | | | transporting bet . . . |
| gbh_m28130 | 2 | 60 | Human interleukin 8 (IL8) gene, complete |
| | | | cds Also knowr as neutrophi . . . |
| uehsf_325_3 | −2 | 19 | Human ROM-K potassium channel protein |
| | | | isoform romk1 mRNA, complete cds |
| uehsf_325_2 | −2 | 19 | Human ROM-K potassium channel protein |
| | | | isoform romk1 mRNA complete cds |
| gbh_u65406_1 | −2 | 19 | Human alternatively spliced potassium |
| | | | channels ROM-K1, ROM-K2. |
| gbh_u65406 | −2 | 19 | Human alternatively spliced potassium |
| | | | channels ROM-K1, ROM-K2. |
| gbh_u77783 | 2 | 17 | Homo sapiens N-methyl-D-aspartate |
| | | | receptor 2D subunit precursor |
| gbh_m69296 | 2 | 17 | Human estrogen receptor-related protein |
| | | | (variant ER from breast |
| uehsf_1158_1 | 2 | 17 | Human estrogen receptor mRHA, |
| | | | complete cds SIM estrogen receptor 0.0 |
| gbh_u535831 | 2 | 17 | Human chromosome 17 cosmid ICRF |
| | | | 105cF06137 olfactory receptor gene |
| gbh_af145029 | −2 | 14 | Homo sapiens transportin-SR (TRN-SR) |
| | | | mRNA, complete cds. |
| gbh_aj133769 | −2 | 14 | Homo sapiens mRNA for nuclear transport |
| | | | receptor |
| gbh_u26209 | 2 | 15 | Human renal sodium/dicarboxylate |
| | | | cotransporter (NADC1) mRNA. |
| uehsf_28080_0 | 2 | 15 | Human renal sodium SIM sodium/ |
| | | | dicarboxylate cotransporter, renal 0.0 |
| gbh_ab026584 | −2 | 14 | Homo sapiens gene for endothelial protein |
| | | | C receptor, complete cds |
| gbh_af106202 | −2 | 14 | Homo sapiens endothelial cell protein |
| | | | C receptor precursor (EP CR) |
| uehsf_1552_0 | −2 | 14 | HSC25E121 Homo sapiens cDNA SIM |
| | | | C/activated protein C receptor, |
| | | | endothelial 0.0 |
| gbh_135545 | −2 | 14 | Homo sapiens endothelial cell protein |
| | | | C/APC receptor (EPCR) mRNA. |
| gbh_af026535 | 2 | 14 | Homo sapiens chemokine receptor |
| | | | (CCR3) mRNA, complete cds. |

Differentially regulated genes were also grouped by their cellular functions such as translational control and protein synthesis, cell cycle control, signal transduction, and metabolism. The results are summarized in Tables 3-7. Table 3 shows a list of genes that are translationally downregulated after IL-α treatment. These genes are mostly involved in cellular protein synthesis. One of the examples is ribosomal protein S4, which is shown to be translationally downregulated with IL-α exposure (Zong et al, PNAS 96:10632-10636, 1999). Among the confirmed genes, the ribosomal protein S4 is a known example of an RNA binding protein (Hershey et al., Translational Control. Cold Spring Harbor Laboratory Press 30:1-29, 1996). Macrophage inflammatory protein-2β is a gene involved in inflammation (Johannes et al., PNAS 96:13118-13123, 1999). Platelet endothelial cell adhesion molecule (PECAM-1), an 15 important gene involved in cellular adhesion, was up-regulated by IL-1α treatment (Mikulits et al., FASEB J. 14:1641-1652, 2000).

TABLE 3
Translationally regulated genes involved in protein synthesis.

| Gene Id | | | | |
|---|---|---|---|---|
| gbh_af097441 | 12 | | | Homo sapiens phenylalanine-tRNA |
| | | | | synthetase (FARS1) mRNA, nuclear |
| uehsf_48978_2 | −4 | | | yj72d01 s1 Homo sapiens cDNA 3″ end |
| | | | | SIM ribosomal protein LB 0.0 |
| uehsf_5730_0 | −4 | | | yh45a10.rl Homo sapiens cDNA, 5″ |
| | | | | end SIM H. sapiens mRNA for |
| | | | | ribosoma . . . |
| uehsf_48374_1 | −2 | | 2 | yj31a10 s1 Homo sapiens cDNA 3″ |
| | | | | end SIM ribosomal protein S4, |
| | | | | X-linke . . . |
| gbh_x57958 | −2 | | 2 | H. sapiens mRNA for ribosomal |
| | | | | protein L7. |
| uehsf_48137_2 | −3 | | | y186e09 r1 Homo sapiens cDNA, 5″ |
| | | | | end SIM ribosomal protein L10 0.0 |
| gbh_j05032 | −3 | | | Human aspartyl-tRNA synthetase |
| uehsf_10195_0 | −3 | | | F3866 Homo sapiens cDNA, 5″ end |
| | | | | SIM aspartyl-tRNA synthetase, alpha |
| gbh_x94754 | −2 | | | H. sapiens mRNA for yeast |
| | | | | methionyl-tRNA synthetase homologue. |
| gbh_ab007155 | −2 | | | Homo sapiens gene for ribosomal |
| | | | | protein S19, partial cds. |
| gbh_x91257 | −2 | | | H. sapiens mRNA for seryl-tRNA |
| | | | | synthetase. |
| gbh_x57959 | −2 | | | H. sapiens mRNA for ribosomal |
| | | | | protein L7. |
| uehsf_722_3 | −2 | | | yg34b06 r1 Homo sapiens cDNA, 5″ |
| | | | | end SIM ribosomal protein S4, |
| | | | | X-linked 0 0 |
| uehsf_48137_1 | −2 | | | yf86e09.r1 Homo sapiens cDNA, 5″ |
| | | | | end SIM ribosomal protein L10 0 0 |
| gbh_49914 | −2 | | | Homo sapiens mRNA for Seryl tRNA |
| | | | | Synthetase, complete cds. |
| uehsf_48136_4 | −2 | | | IB365 Homo sapiens cDNA, 3″ end |
| | | | | SIM ribosomal protein L10 7.4e-214 |
| gbh_m58458 | −2 | [symbol] | | Human ribosomal protein S4(RPS4X) |
| | | | | isoform mRNA, complete cds. |
| gbh_af041428 | −2 | | | Homo sapiens ribosomal protein s4 |
| | | | | X isoform gene, complete cds. |
| gbh_m77234 | −2 | | | Human ribosomal protein S3a mRHA, |
| | | | | complete cds. |

Table 4 lists a group of genes involved in cell signaling. Ribosomal S6 kinase is a gene plays an important role in regulating translation by controlling the biosynthesis of translational components which make up the protein synthetic apparatus (Chu et al., Stem Cells 14:41-46, 1996). This may also explain the high percentage of translationally regulated genes. Table 5 lists a group of genes involved in cell cycle control and apoptosis. Some of them are inhibitors of apoptosis proteins, others are cyclin GI, CDC7 and CDC42. Table 6 shows genes involved in cellular metabolism. One example is dihydrofolate reductase gene, which has been well studied as a gene controlled by translational autoregulation (Bristol et al., J. Immunology 145: 4108-4114, 1990). These results provide further validation of polysome gene expression analysis technology.

TABLE 4
Translationally regulated genes involved in cell signaling.

| Gene Id | | | |
|---|---|---|---|
| gbh_af184965 | 22 | | Homo sapiens ribosomal S6 kinase |
| | | | (RPS6KAB) mRNA, complete cds. |
| uehsf_47562_0 | 9 | | FB21G3 Homo sapiens cDNA, 3″ end |
| | | | SIM ribosomal protein S18 8.9e-210 |
| gbh_ab020236 | 4 | 2 | Homo sapiens gene for ribosomal protein |
| | | | L27A, complete cds |
| gbh_x03342 | 4 | 2 | Human mRNA for ribosomal protein L32. |
| uehsf_29812_6 | 5 | | yg10f02.r1 Homo sapiens cDNA, 5″ end |
| | | | SIM Cyclotella species ribosomal RN . . . |
| gbh_af012072 | 4 | 2 | Homo sapiens eIF4Gll mRNA, complete |
| | | | cds. |
| gbh_x54326 | 3 | −2 | H. sapiens mRNA for glutaminyl-tRNA |
| | | | synthetase |
| gbh_af037447 | 4 | | Homo sapiens ribosomal S6 protein |
| | | | kinase mRNA, complete cds. |
| gbh_ab016869 | 2 | 2 | Homo sapiens mRNA for p70 ribosomal |
| | | | S6 kinase beta, complete cds. |
| gbh_aj012375 | 2 | 2 | Homo sapiens mRNA for SUl1 protein |
| | | | translation initiation factor. |
| gbh_al121586_3 | 2 | −2 | Human DNA sequence from clone |
| | | | RP3-47704 on chromosome 20. |
| | | | Contains ESTs . . . |
| gbh_al031777_7 | 2 | 2 | Human DNA sequence from clone 34820 |
| | | | on chromosome 6p21.31-22.2. |
| | | | Contain . . . |
| gbh_al031777_10 | 2 | −2 | Human DNA sequence from clone 34820 |
| | | | on chromosome 6p21.31-22.2. |
| | | | Contain . . . |
| uehsf_36282_0 | 2 | 2 | yj60f03 s1 Homo sapiens cDNA, 3″ end |
| | | | SIM acidic ribosomal protein P1 |
| gbh_s80343 | 2 | 2 | Arg RS = arginyl-t RNA synthetase |
| | | | [human, ataxia-telangiectasia patients . . . |
| gbh_af173378 | 2 | 2 | Homo sapiens 60S acidic ribosomal |
| | | | protein PO mRNA, complete cds |
| gbh_x63527 | 3 | | H. sapiens mRNA for ribosomal |
| | | | protein L19. |
| uehsf_2042_3 | 3 | | yh20h10.r1 Homo sapiens cDNA 5″ end |
| | | | SIM ribosomal protein L19 1 2e-297 |
| uehsf_36509_0 | 3 | | HUM024C03A Homo sapiens cDNA 3″ |
| | | | end SIM 40 S RIBOSOMAL PROTEIN |
| | | | S12. [dbEST . . . |

TABLE 5
Translationally regulated genes involved
in cell cycle control and apoptosis.

| Gene Id | | | |
|---|---|---|---|
| gbh_u45878 | 20 | 2 | Human inhibitor of apoptosis protein 1 |
| | | | mRNA, complete cds. |
| gbh_af128625 | 16 | 2 | Homo sapiens CDC42-binding protein |
| | | | kinase beta (CDC42BPB) mRNA. |
| gbh_d28540 | 9 | 2 | Human mRNA for Diff6, H5, CDC10 |
| | | | homologue, complete cds |
| gbh_af015592 | 5 | 2 | Homo sapiens Cdc7 (CDC7) mRNA, |
| | | | complete cds. |
| gbh_y11593 | 4 | 2 | Homo sapiens mRNA for peanut-like |
| | | | protein 1, PNUTL1 (hCDCrel-1). |
| gbh_af006988 | 4 | 2 | Homo sapiens septin (CDCrel-1) gene, |
| | | | alternatively spliced. |
| gbh_u74628 | 4 | 2 | Homo sapiens cell division control related |
| | | | protein (hCDCrel-1) |
| gbh_af006988_1 | 4 | 2 | Homo sapiens septin (CDCrel-1) gene, |
| | | | alternatively spliced. |
| gbh_u94507 | 3 | 2 | Human lymphocyte associated receptor of |
| | | | death 6 mRNA, alternatively |
| uehsf_5550_1 | 3 | 2 | yf91g10.r1 Homo sapiens cDNA, 5″ end |
| | | | SIM hypothetical protein, CDC1 . . . |
| qbh_z75311 | 3 | −2 | H sapiens mRNA for RAD50 |
| gbh_u61836 | 2 | 2 | Human putative cyclin G1 interacting |
| | | | protein mRNA, partial |
| uehsf_47046_1 | 2 | 2 | yh19g10.r1 Homo sapiens cDNA, 5″ end |
| | | | SIM senne/threonine kinase stk1 |
| gbh_x79193 | 2 | 2 | H. sapiens CAK mRNA for CDK-activating |
| | | | kinase. |
| gbh_x77743 | 2 | 2 | H. sapiens CDK activating kinase mRNA |
| gbh_x77303 | 2 | 2 | H. sapiens CAK1 mRNA for Cdk-activating |
| | | | kinase. |
| gbh_af228149 | 2 | −2 | Homo sapiens from Nu-6 cyclin-dependent |
| | | | kinase 2 interacting |
| uehsf_3809_0 | 2 | 2 | zb65e01 s1 Homo sapiens cDNA, 3″ end |
| | | | SIM Mus musculus cycli. |
| gbh_af228148 | 2 | −2 | Homo sapiens from HeLa cyclin-dependent |
| | | | kinase 2 interacting |

TABLE 6
Translationally regulated genes involved in metabolism.

| Gene Id | | | |
|---|---|---|---|
| uehsf_39110_3 | −6 | 2 | HSB95G072 Homo sapiens cDNA SIM ATP |
| | | | synthase, alpha subunit, mitochondria . . . |
| gbh_k01612 | −6 | | Human dihydrofolate reductase gene, |
| | | | exons 1 and 2. |
| gbh_j00140 | −6 | | Human dihydrofolate reductase gene. |
| gbh_aj001541 | −5 | 2 | Homo sapiens peroxisomal branched chain |
| | | | acyl-CoA oxidase gene. |
| gbh_x95190 | −5 | 2 | H. sapiens mRNA for Branched chain |
| | | | Acyl-CoA Oxidase. |
| gbh_I19501 | −4 | 2 | Homo sapiens (clone pGHSCBS) |
| | | | cystathionine beta-synthase subunit |
| gbh_af121202 | −4 | −2 | Homo sapiens methionine synthase |
| | | | reductase (MTRR) gene, exon 1 and |
| gbh_af121214 | −4 | −2 | Homo sapiens methionine synthase |
| | | | reductase (MTRR) mRNA complete |
| gbh_af151538 | −4 | 2 | Homo sapiens deoxycytidyl transferase |
| | | | (REV1) mRNA, complete cds. |
| gbh_aj001050 | −4 | 2 | Homo sapiens thioredoxin reductase |
| gbh_af208018 | −4 | 2 | Homo sapiens thioredoxin reductase |
| | | | (TR) mRNA, complete cds. |
| uehsf_88_0 | −4 | 2 | Human famesyl pyrophosphate synthetase |
| | | | mRNA (hpt807). 3″ end SIM famesy . . . |
| gbh_x59617 | −4 | −2 | H. sapiens RR1 mRNA for large subunit |
| | | | ribonucleotide reductase |
| gbh_x59543 | −4 | −2 | Human mRNA for M1 subunit of |
| | | | ribonucleotide reductase. |
| gbh_af107045 | −4 | −2 | Homo sapiens ribonucleotide reductase M1 |
| | | | subunit (RRM1) gene. |
| uehsf_2037_0 | −4 | −2 | H. sapiens RR1 mRNA for large subunit |
| | | | ribonucleotide reductase SI . . . |
| gbh_u24267 | −3 | 2 | Human pyrroline-5-carboxylate |
| | | | dehydrogenase (P5CDh) mRNA, short |
| gbh_u80040 | −3 | −2 | Human nuclear aconitase mRNA, encoding |
| | | | mitochondrial protein. |
| gbh_af037601 | −3 | −2 | Homo sapiens leucine carboxyl |
| | | | methyltransferase (LCMT) mRNA. |

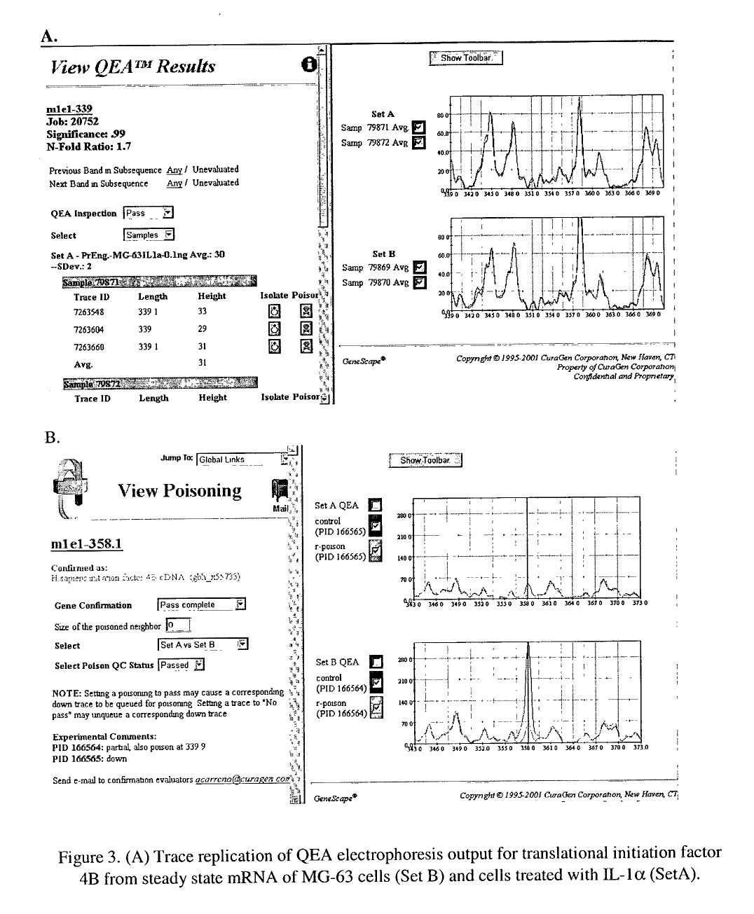

FIG. 3 shows representative replication QEA traces for translational initiation factor 4B. Shown is the polysome distribution of cellular mRNAs in MG-63 control cells (FIG. 3A) and cells treated with IL-1α for 6 hr (FIG. 3B). FIG. 3A shows trace replication of QEA electrophoresis output for translational initiation factor 4B from steady state mRNA of MG-63 cells (Set B) and cells treated with IL-la (SetA). FIG. 3B shows poisoned QEA electrophoresis output from polysome isolated mRNA of MG-63 cells (Set B) and cells treated with IL-1α (Set A). Traces are expression profile before poisoning and after poisoning. The total mRNA expression level for translational initiation factor 4B showed no difference based upon steady state mRNA gene expression analysis studies (FIG. 3A). However, the level of actively translated forms of translational initiation factor 4B was significantly down regulated in MG-63 cells treated with IL-1α compared with control MG-63 cells (FIG. 3B). Translational initiation factor 4B plays a critical role in regulating a global translation initiation, and this may explain the fact that over 40% of the genes are regulated to different degrees by translation regulation (Sheikh et al., Oncogene 18:6121-6128, 1999). There are many other genes that are translationally regulated such as thymidylate synthase (Sachs et al., Cell 89:831-8, 1997) and p53 (Ruan et al., Analysis of mRNA Formation and Function, Academic Press, 305-321, 1997).

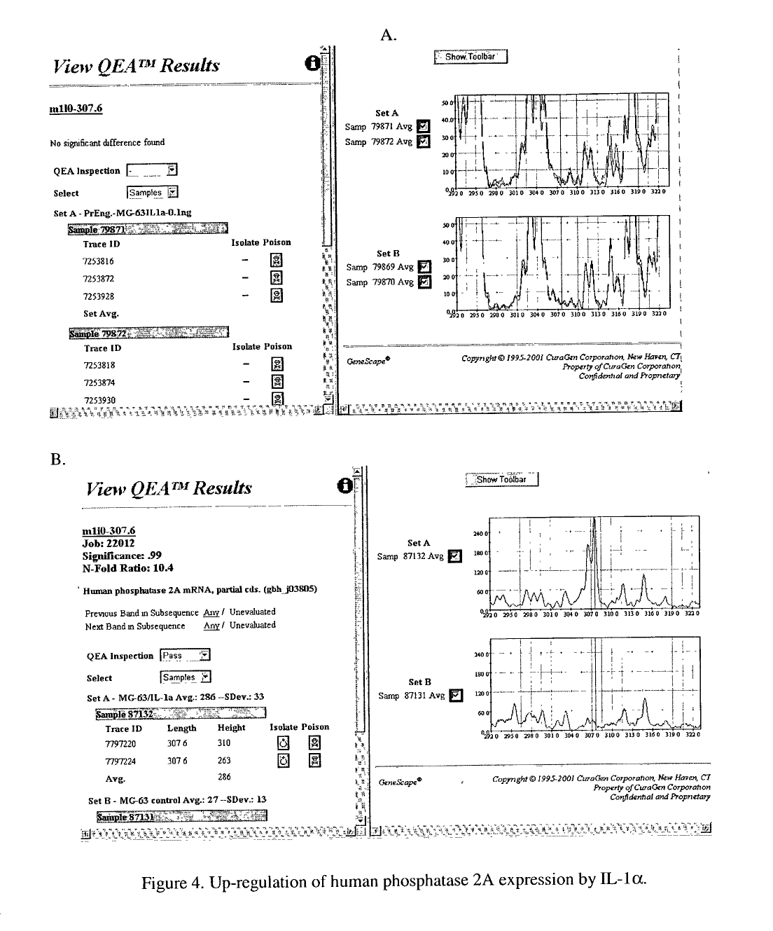

Another known translationally regulated gene is phosphatase type 2A (PP2A; Baharians et al., J. Biol. Chem. 273: 19019-24, 1998). The expression of phosphatase type 2A was identical in MG-63 control cells and cells treated with IL-1α based upon steady state level of mRNA expression (FIG. 4A). FIG. 4A shows trace replication of QEA electrophoresis output for phosphatase 2A from total mRNA of MG-63 control cells (Set B.) and cells treated with IL-1α (Set A). FIG. 4B shows trace replication of QEA electrophoresis output for phosphatase 2A from polysomal isolated mRNA of MG-63 control cells (Set B) and cells treated with IL-1α (Set A). Phosphatase type 2A expression level was significantly up-regulated by nearly 10-fold after IL-1α exposure based upon polysomal isolated actively translated mRNA (FIG. 4B). It has been shown that in the mouse fibroblast cell line NIH3T3, the catalytic subunit of PP2A is subject to a potent autoregulatory mechanism that adjusts PP2A protein to constant levels. This control is exerted at the translational level and does not involve regulation of transcription or RNA processing. Protein phosphatase 2A is involved in MAP kinase signal-transduction pathways. It has been suggested that protein phosphatase 2A plays an important role in response to IL-6 during acute phase responses and inflammation (Choi et al., Immunol. Lett. 61: 103-107, 1998). These results, taken together, suggest that IL-1α regulates protein phosphatase 2A as part of the signaling event in MG-63 cells.

Table 7 shows the confirmed genes that were translationally regulated in MG-63 cells treated with IL-1α. One of the genes is calcium modulating cyclophilin ligand (CAML). CAML was originally described as a cyclophilin B-binding protein whose overexpression in T cells causes a rise in intracellular calcium, thus activating transcription factors responsible for the early immune response (Chu et al., Stem Cells 14:41-46). CAML is an ER membrane bound protein and oriented toward cytosol (Rousseau et al., PNAS 93:1065-1070, 1996). It was shown that CAML functions as a regulator to control Ca²⁺ storage (Bram et al., Nature 371:355-358, 1994). The steady state level of CAML mRNA in both controlling MG-63 and MG-63 treated with IL-1α was no difference. However, the polysome isolated, actively translated mRNA in MG-63 cells treated with IL-1α was down regulated by nearly 4 fold.

TABLE 7
Translational regulated gene list confirmed with poisoning experiment.

| Gene Id | | | | |
|---|---|---|---|---|
| gbh_x55733 | −9 | [symbol] | | H sapiens initiation factor 4B cDNA. |
| gbh_d30655 | −4 | [symbol] | −1 | Homo sapiens mRNA for eukaryotic |
| | | | | initiation factor 4AII (eIF4A-II), complete |
| gbh_x56794 | −4 | | | H sapiens CD44R mRNA. |
| gbh_m58458 | −2 | [symbol] | | Human ribosomal protein S4 (RPS4X) |
| | | | | isoform mRNA, complete cds |
| gbh_x60489 | −2 | [symbol] | | Human mRNA for elongation |
| | | | | factor 1 beta. |
| gbh_af068179 | −4 | [symbol] | 2 | Homo sapiens calcium modulating |
| | | | | cyclophilin ligand CAMLG (CAMLG) |
| gbh_x53800 | 7 | [symbol] | −2 | Human mRNA for macrophage |
| | | | | inflammatory protein-2beta (MIP2beta) |
| gbh_m31166 | 3 | [symbol] | 2 | Human tumor necrosis factor-inducible |
| | | | | protein (aka pentaxin-related protei |

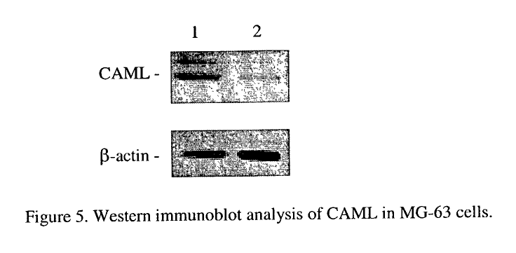

The western iminunoblot for CAML confirmed that indeed the protein level of CAML in MG-63 cells treated with IL-1α was down regulated as well. as is shown in FIG. 5. Cytosolic extracts from MG-63 (lane 1) and MG-63 cells treated with IL-1α (lane 2) were prepared. CAML protein was detected by immunoblot analysis by using an anti-CAML polyclonal antibody. Filtered membranes were then reprobed with an anti-β-actin monoclonal antibody to control for loading and integrity of protein.

### EXAMPLE 3

### Microsomal Enrichment of Actively Translated mRNAs Encoding for Secreted or Membrane-associated Proteins

Materials

Materials used are Listed in Table 8.

TABLE 8
Materials used in microsome mRNA enrichment

| Reagents/Material | Vendor | Stock Number |
|---|---|---|
| TK150 M * | | |
| Sucrose | Sigma | S-0389 |
| 0.8 M sucrose * | | |
| 1.3 M sucrose * | | |
| 2.05 M sucrose * | | |
| 2.5 M sucrose * | | |
| Heprin | Gibco BRL | 15077-019 |
| Superaseln | Ambion | 2696 |
| 2-mercaptoethanol | Sigma | M7154 |
| Falcon tube (15 ml) | | |
| RNase Zap | Ambion | 9780 |
| Homogenizer | Glas-Col | |
| tube and pestle set | Glas-Col | 099C S440 |
| DEPC-water | Ambion | 9922 |
| Beckman centrifuge tubes (17 ml) | Beckman | 344061 |

Methods
Preparing Pestles and Tubes:
Use RNase Zap to zap cleaned Teflon pestle and tube sets, followed by rinsing with DEPC treated water.
Set Teflon pestles and tubes on ice.
   Preparing Tissues:
Fresh mouse tissue were carefully minced with scalpel and then soaked with soaking buffer containing 1001 μg/ml of cycloheximide for 10 minutes. Buffer then removed and tissue sample will then be snap freeze with liquid nitrogen.
   Homogenizing Tissues:
Retrieve tissues from −80 C. freezer and put them on ice.
Add 1 ml of homogenizing buffer into each tissue sample.
Transfer tissues in homogenizing buffer into Teflon tube and leave the tubes on ice.
Set the homogenizer at speed setting of 30, homogenize tissue sample for 5 strokes, and then set the homogenizer at speed setting of 75 for another 10 strokes. Note: During homogenizing, leave the teflon tubes on ice all the time. Make sure that samples are well homogenized without any noticeable chunks.
Transfer the lysates into a new set of RNase free eppendorf tubes and centrifuge at 13,200 rpm for 10 minutes to pellet nuclei.
During the centrifugation, pipette 5.5 ml of 2.5M sucrose (in TK150M) into 5 ml Falcon tubes.
After the spin is done, pipette out 1 ml supernatant into the Falcon tube containing 5.5 ml of 2.5M sucrose. If the supernatant is less than 1 ml, add extra 0.8M sucrose to make up the volume. If more than 1 ml, just take 1 ml.
Vortex Falcon tubes well. The final concentration of sucrose should be 2.1M.
   Homogenizing Cell Culture Samples:
2 ×10⁸ culture human melanoma HepG2, HS688 (A) and HS688 (B) cells were incubated with 100 μg/ml cycloheximide for 10 min.
Remove media and scrap off cells in 10 ml ice-cold Ix PBS with 100 jig/ml cycloheximide.
   Spin at 1500 rpm for 4 min. to pellet cells, then wash pellets twice with (30 ml) ice-cold PBS containing 100 μg/ml cycloheximide.
   Cells were allowed to swell for 5 min. in 1 ml ice-cold RSB buffer (10 mM KCl, 1.5 mM MgCl₂, and 10 mM Tris-HCl at pH 7.4) plus 1 mg/ml heparin. Mechanically rupture cells with 10 strokes of dounce glass homogenizer. Monitor cells rupture by trypan blue (0.05%) in saline.
   Transfer the homogenate into a new set of RNase free eppendorf tubes and spin at 3000 rpm for 2 min. at 4° C. Save the supernatant.
   After the spin is done, pipette out 1 ml supernatant into the Falcon tube containing 5.5 ml of 2.5M sucrose. If the supernatant is less than 1 ml, add extra 0.8M sucrose to make up the volume. If more than 1 ml, just take 1ml.
   Vortex Falcon tubes well. The final concentration of sucrose should be 2.1M.
      Preparing Sucrose Gradient:
   Take a new set of 17 ml centrifuge tubes and add 2 ml of 2.5M sucrose (in TK150M).
   Layer the sample extract (in the final concentration of 2.1M sucrose) on the top of the 2.5 M sucrose phase.
   Then slowly pipette 6.5 ml of 2.05 M sucrose (in TK150M).
   Add another 2 ml layer of 1.3M sucrose (in TK150M).
   Weigh and balance the samples well with addition of 1.3M sucrose solution.
      Ultra-centrifugation:
   Turn on the Ultracentrifuge (before starting tissue homogenization step). Also set the temperature of ultra centrifugation at 4° C. and leave the vacuum on.
   Weigh and balance well the samples with addition of 1.3M sucrose solution.
   Set the sample tubes into brackets and carefully screw on the top caps.
   Take the rotor out of the centrifuge.
   Set the brackets with samples onto the SW28 rotor and mount the rotor back into the centrifuge. (Please align the rotor well!)
   Check the ultracentrifugating parameters:
      Speed: 25000
      Time: 5 hours
      Temp: 4° C.
   Hit the start key.
   After the centrifugation is done, hit the vacuum button to release vacuum.
   Take the SW28 rotor out of the centrifuge.
   Remove the brackets from the rotor.
   Open the cap of the brackets and take out the Beckman centrifuge tubes.
   Carefully pipette out 10 fractions per sample, 1 ml each, into a new set of RNase free eppendorf tubes (leave tubes on ice).
   Aliquot 10 μl of samples from each fraction and dilute samples with water to 1:20 and check OD at 260 nm.
   Store samples in eppendorf tubes at −80C.
   Mount rotor back into the centrifuge and turn off the power.
   Record the use of ultracentrifuge into the logbook.
      Reagent Preparation
   TK150M buffer: (150 mM KCl, 5 mM MgCl₂, 50 mM Tris-HCl at pH 7.5)

To make 500 ml of TK150M buffer: Add
1M KCl: 75 ml
1M MgCl₂: 2.5 ml
Tris-HCl (PH7.5): 25 ml
DEPC H₂O: 397.5 ml

Filter the solution and store at room temperature.
2.5M sucrose in TK 150M buffer (Filter the solution and store at 4° C.)
2.05M sucrose in TK150M buffer (Filter the solution and store at 4° C.)
1.3M sucrose in TK 150M buffer (Filter the solution and store at 4° C.)
0.8M sucrose in TK150M buffer (Filter the solution and store at 4° C.)
   Homogenizing Buffer (Make Within the Same Day of Use):

Add 50 ul of b-ME and 20 ul of Superaseln (RNase inhibitor) for 1 ml of homogenizing buffer.

Soaking buffer: 50 mM HEPES buffer pH 7.4, 250 mM NaCl, 10 mM MgCl₂ with RNase inhibitor and 100 mg/ml cyclohexamide (all final concentrations).

Results

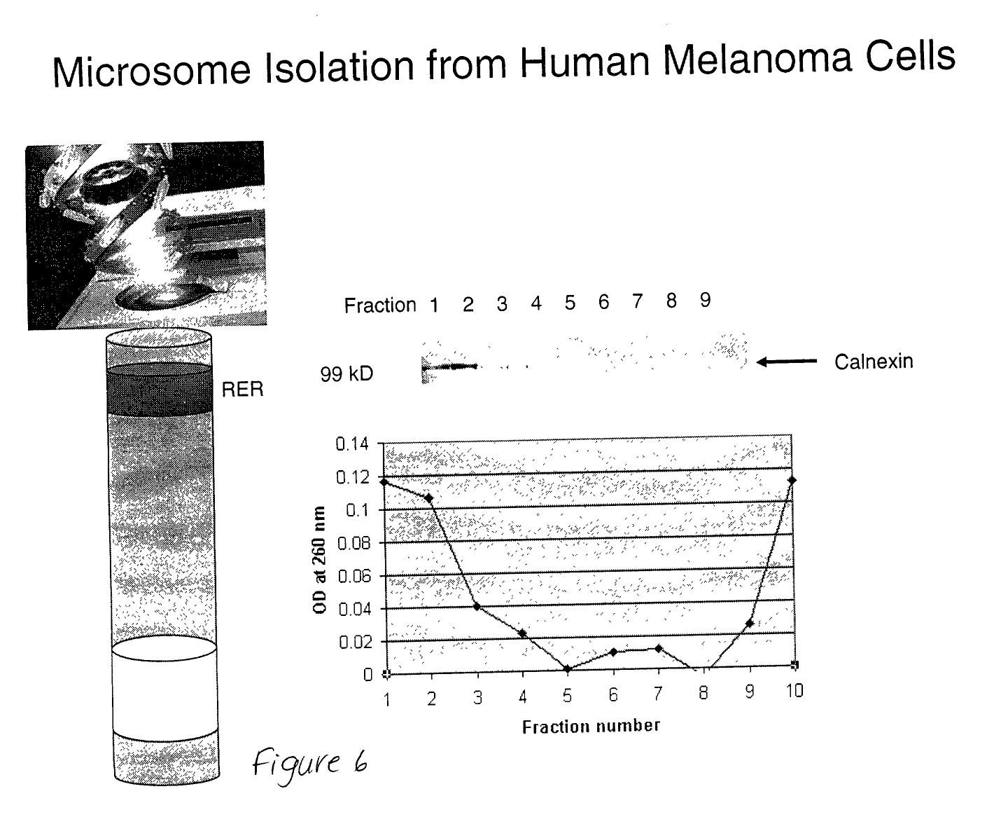

Microsomes were isolated using sucrose gradient centrifugation as described above. Samples were then processed for Western immunoblot analysis for the rough ER marker protein calnexin. FIG. 6 demonstrates enrichment of microsomes in fractions 1 and 2. Table 9 lists genes from a random sequencing of 50 microsomally derived cDNA clones; 80% of the genes arc either secreted or membrane-bound genes.

Using microsomal enrichment and SeqCalling™ technology, 7000 unique genes were identified and among them, 80% of the 7000 genes were secreted and/or membrane bound genes.

TABLE 9

Membrane Bound/Secretory Pathway
Urokinase receptor-associated protein uPARAP
Adhesion molecule (CD44) fibrillin
Toll-like receptor 2 type1
Human collagenase type IV
Tapasin (NGS-17)
Calreticulin
Translocon-associated protein alpha
Secreted
Vascular endothelial growth factor (VEGF)
Human procollegen type I alpha-2 chain
Heparan sulfate proteoglycan (HSPG2)
Human growth hormone-dependent
insulin-like growth factor-binding
protein mRNA
Cytoplasmic
Homo sapiens putative oral tumor
suppressor protein (doc-1)
Bruton's tyrosine kinase (BTK)
Unknown Function
KIAA1149 protein
Patent EP0892047-unidentified
U.S. Pat. No. 5,858,674-unknown
Patent EP0892047-unidentified
FLJ23084 fis

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. A method for identifying, classifying, or quantifying one or more nucleic acids in a sample comprising a plurality of nucleic acids having different nucleotide sequences, said method comprising:
(a) providing a CDNA sample prepared from a population of microsomes;
(b) probing said sample with one or more recognition means, each recognition means recognizing a different target nucleotide subsequence or a different set of target nucleotide subsequences;
(c) generating one or more output signals from said sample probed by said recognition means, each output signal being produced from a nucleic acid in said sample by recognition of one or more target nucleotide subsequences in said nucleic acid by said recognition means and comprising a representation of (i) the length between occurrences of target nucleotide subsequences in said nucleic acid, and (ii) the identities of said target nucleotide subsequences in said nucleic acid or the identities of said sets of target nucleotide subsequences among which are included the target nucleotide subsequences in said nucleic acid; and
(d) searching a nucleotide sequence database to determine sequences that are predicted to produce or the absence of any sequences that are predicted to produce said one or more output signals produced by said nucleic acid, said database comprising a plurality of known nucleotide sequences of nucleic acids that may be present in the sample, a sequence from said database being predicted to produce said one or more output signals when the sequence from said database has both (i) the same length between occurrences of target nucleotide subsequences as is represented by said one or more output signals, and (ii) the same target nucleotide subsequences as are represented by said one or more output signals, or target nucleotide subsequences that are members of the same sets of target nucleotide subsequences represented by said one or more output signals, whereby said one or more nucleic acids in said sample are identified, classified, or quantified.

2. The method of claim 1 wherein each recognition means recognizes one target nucleotide subsequence, and wherein a sequence from said database is predicted to produce a particular output signal when the sequence from said database has both the same length between occurrences of target nucleotide subsequences as is represented by the output signal and the same target nucleotide subsequences as represented by the particular output signal.

3. The method of claim 1 wherein each recognition means recognizes a set of target nucleotide subsequences, and wherein a sequence from said database is predicted to produce a particular output signal when the sequence from said database has both the same length between occurrences of target nucleotide subsequences as is represented by the particular output signal, and the target nucleotide subsequences are members of the sets of target nucleotide subsequences represented by the particular output signal.

4. The method of claim 1 further comprising dividing said sample of nucleic acids into a plurality of portions and performing the steps of claim 1 individually on a plurality of said portions, wherein a different one or more recognition means are used with each portion.

5. The method of claim 1 wherein the quantitative abundances of nucleic acids in said sample are determined from the quantitative levels of the output signals produced by said nucleic acids.

6. The method of claim 7 wherein the cDNA is prepared from a plant, a single celled animal, a multicellular animal, a bacterium, a virus, a fungus, or a yeast.

7. The method of claim 6 wherein the CDNA is prepared from a mammal.

8. The method of claim 6 wherein the mammal is a human.

9. The method of claim 6 wherein said database comprises substantially all the known expressed sequences of said plant, single celled animal, multicellular animal, bacterium, virus, fungus, or yeast.

10. The method of claim 7 wherein the cDNA is of total cellular RNA or total cellular poly(A) RNA.

11. The method of claim 6 wherein the recognition means are one or more restriction endonucleases whose recognition sites are said target nucleotide subsequences, and wherein the step of probing comprises digesting said sample with said one or more restriction endonucleases into fragments and ligating double stranded adapter DNA molecules to said fragments to produce ligated fragments, each said adapter DNA molecule comprising (i) a shorter stand having no 5′ terminal phosphates and consisting of a first and second portion, said first portion at the 5′ end of the shorter strand and being complementary to the overhang produced by one of said restriction endonucleases, and (ii) a longer strand having a 3′ end subsequence complementary to said second portion of the shorter strand; and wherein the step of generating further comprises melting the shorter strand from the ligated fragments, contacting the ligated fragments with a DNA polymerase, extending the ligated fragments by synthesis with the DNA polymerase to produce blunt-ended double stranded DNA fragments, and amplifying the blunt-ended fragments by a method comprising contacting the blunt-ended fragments with the DNA polymerase and primer oligodeoxynucleotides, said primer oligodeoxynucleotides comprising a hybridizable portion of the sequence of the longer strand of the adapter nucleic acid molecule, and said contacting being at a temperature not greater than the melting temperature of the primer oligodeoxynucleotide from a strand of the blunt-ended fragments complementary to the primer oligodeoxynucleotide and not less than the melting temperature of the shorter strand of the adapter nucleic acid molecule from the blunt-ended fragments.

12. The method of claim 6 wherein the recognition means are one or more restriction endonucleases whose recognition sites are said target nucleotide subsequences, and wherein the step of probing further comprises digesting the sample into fragments with said one or more restriction endonucleases.

13. The method of claim 12 further comprising:
(a) identifying a fragment of a nucleic acid in the sample which generates said one or more output signals; and
(b) recovering said fragment.

14. The method of claim 13 wherein the output signals generated by said recovered fragment are not predicted to be produced by a sequence in said nucleotide sequence database.

15. The method of claim 13 which further comprises using at least a hybridizable portion of said recovered fragment as a hybridization probe to bind to a nucleic acid.

16. The method of claim 12 wherein the step of generating further comprises after said digesting: removing from the sample both nucleic acids which have not been digested and nucleic acid fragments resulting from digestion at only a single terminus of the fragments.

17. The method of claim 16 wherein prior to digesting, the nucleic acids in the sample are each bound at one terminus to a biotin molecule, and said removing is carried out by a method which comprises contacting the nucleic acids in the sample with streptavidin or avidin affixed to a solid support.

18. The method of claim 16 wherein prior to digesting, the nucleic acids in the sample are each bound at one terminus to a hapten molecule, and said removing is carried out by a method which comprises contacting the nucleic acids in the sample with an anti-hapten antibody affixed to a solid support.

19. The method of claim 12 wherein said digesting with said one or more restriction endonucleases leaves single-stranded nucleotide overhangs on the digested ends.

20. The method of claim 19 wherein the step of probing further comprises hybridizing double-stranded adapter nucleic acids with the digested sample fragments, each said double-stranded adapter nucleic acid having an end complementary to said overhang generated by a particular one of the one or more restriction endonucleases, and ligating with a ligase a strand of said double-stranded adapter nucleic acids to the 5′ end of a strand of the digested sample fragments to form ligated nucleic acid fragments.

21. The method of claim 20 wherein said digesting with said one or more restriction endonucleases and said ligating are carried out in the same reaction medium.

22. The method of claim 21 wherein said digesting and said ligating comprises incubating said reaction medium at a first temperature and then at a second temperature, wherein said one or more restriction endonucleases are more active at the first temperature than the second temperature and said ligase is more active at the second temperature than the first temperature.

23. The method of claim 22 wherein said incubating at said first temperature and said incubating at said second temperature are performed repetitively.

24. The method of claim 20 wherein the step of probing further comprises prior to said digesting: removing terminal phosphates from DNA in said sample by incubation with an alkaline phosphatase.

25. The method of claim 24 wherein said alkaline phosphatase is heat labile and is heat inactivated prior to said digesting.

26. The method of claim 20 wherein said generating step comprises amplifying the ligated nucleic acid fragments.

27. The method of claim 26 wherein said amplifying is carried out by use of a nucleic acid polymerase and primer nucleic acid strands, said primer nucleic acid strands comprising a hybridizable portion of the sequence of said strands ligated to said sample fragments.

28. The method of claim 27 wherein the primer nucleic acid strands have a G+C content of between 40% and 60%.

29. The method of claim 27 wherein each said double-stranded adapter nucleic acid comprises a shorter strand hybridized to a longer strand, wherein the longer strand is said strand of said double-stranded adapter nucleic acid that becomes ligated to the digested sample fragments, wherein each said shorter strand is complementary both to one of said single-stranded nucleotide overhangs and to one of said longer strands, and said generating step comprises prior to said amplifying step the melting of the shorter strand from the ligated fragments, contacting the ligated fragments with a DNA polymerase, extending the ligated fragments by synthesis with the DNA polymerase to produce blunt-ended double stranded DNA fragments, and wherein the primer nucleic acid strands comprise a hybridizable portion of the sequence of said longer strands.

30. The method of claim 27 wherein each said double-stranded adapter nucleic acid comprises a shorter strand hybridized to a longer strand, wherein the longer strand is said strand of said double-stranded adapter nucleic acid that becomes ligated to the digested sample fragments, wherein each said shorter strand is complementary both to one of said single-stranded nucleotide overhangs and to one of said longer strands, and said generating step comprises prior to said amplifying step the melting of the shorter strand from the ligated fragments, contacting the ligated fragments with a DNA polymerase, extending the ligated fragments by synthesis with the DNA polymerase to produce blunt-ended double stranded DNA fragments, and wherein the primer nucleic acid strands comprise the sequence of said longer strands.

31. The method of claim 30 wherein during said amplifying step the primer nucleic acid strands are annealed to the ligated nucleic acid fragments at a temperature that is less than the melting temperature of the primer nucleic acid strands from strands complementary to the primer nucleic acid strands but greater than the melting temperature of the shorter adapter strands from said blunt-ended fragments.

32. The method of claim 30 wherein the primer nucleic acid strands further comprise at the 3′ end of and contiguous with the longer strand sequence, the sequence of the portion of the restriction endonuclease recognition site remaining on a nucleic acid fragment terminus after digestion by the restriction endonuclease.

33. The method of claim 32 wherein each said primer nucleic acid strand further comprises at its 3′ end one or more additional nucleotides 3′ to and contiguous with said sequence of the portion of the restriction endonuclease recognition site remaining on a nucleic acid fragment after digestion by said restriction endonuclease, whereby the ligated nucleic acid fragment amplified is that comprising said remaining portion of said restriction endonuclease recognition site contiguous to said one or more additional nucleotides.

34. The method of claim 33 wherein said primer nucleic acid strands are detectably labeled, such that said primer nucleic acid strands comprising a particular said one or more additional nucleotides can be detected and distinguished from said primer nucleic acid strands comprising a different said one or more additional nucleotides.

35. The method of claim 6 wherein the recognition means comprise oligomers of nucleotides, universal nucleotides, nucleotide-mimics, or a combination of nucleotides, universal nucleotides, and nucleotide-mimics, said oligomers being hybridizable with the target nucleotide subsequences.

36. The method of claim 35 wherein the step of generating comprises amplifying with a nucleic acid polymerase and with primers, the sequence of said primers comprising (i) the sequence of said oligomers, and (ii) an additional subsequence 5′ to said sequence of said oligomers.

37. The method of claim 36 further comprising:
(a) identifying a fragment of a nucleic acid in the sample which generates said one or more output signals; and
(b) recovering said fragment.

38. The method of claim 37 wherein said one or more output signals generated by said recovered fragment are not predicted to be produced by any sequence in said nucleotide database.

39. The method of claim 37 which further comprises using at least a hybridizable portion of said recovered fragment as a hybridization probe to bind to a nucleic acid.

40. The method of claim 1 wherein said one or more output signals further comprise a representation of whether an additional target nucleotide subsequence is present in said nucleic acid in the sample between said occurrences of target nucleotide subsequences.

41. The method of claim 40 wherein said additional target nucleotide subsequence is recognized by a method comprising contacting nucleic acids in the sample with oligomers of nucleotides, nucleotide-mimics, or mixed nucleotides and nucleotide-mimics, which are hybridizable with said additional target nucleotide subsequence.

42. The method of claim 1 wherein the step of generating comprises generating said one or more output signals only when an additional target nucleotide subsequence is not present in said nucleic acid in the sample between said occurrences of target nucleotide subsequences, and wherein a sequence from said sequence database is predicted to produce said one or more output signals when the sequence from said database (i) has the same length between occurrences of target nucleotide subsequences as is represented by said one ore more output signals, (ii) has the same target nucleotide subsequences as are represented by said one or more output signals, or target nucleotide subsequences that are members of the same sets of target nucleotide subsequences as are represented by said one or more output signals and (iii) does not contain said additional target nucleotide subsequence between occurrences of said target nucleotide subsequences.

43. The method of claim 42 wherein the step of generating comprises amplifying nucleic acids in the sample, and wherein said additional target nucleotide subsequence is recognized by a method comprising contacting nucleic acids in the sample with (a) oligomers of nucleotides, nucleotide-mimics, or mixed nucleotides and nucleotide-mimics, which hybridize with said additional target nucleotide subsequence and disrupt the amplifying step; or (b) restriction endonucleases which have said additional target nucleotide subsequence as a recognition site and digest the nucleic acids in the sample at the recognition site.

44. The method claim 12 wherein the step of generating further comprises separating nucleic acid fragments by length.

45. The method of claim 44 wherein the step of generating further comprises detecting said separated nucleic acid fragments.

46. The method of claim 45 wherein the abundance of a nucleic acid comprising a particular nucleotide sequence in the sample is determined from the level of the one or more output signals produced by said nucleic acid that are predicted to be produced by said particular nucleotide sequence.

47. The method of claim 45 wherein said detecting is carried out by a method comprising staining said fragments with silver, labeling said fragments with a DNA intercalating dye, or detecting light emission from a fluorochrome label on said fragments.

48. The method of claim 45 wherein said representation of the length between occurrences of target nucleotide subsequences is the length of fragments determined by said separating and detecting steps.

49. The method of claim 45 wherein said separating is carried out by use of liquid chromatography or mass spectrometry.

50. The method of claim 45 wherein said separating is carried out by use of electrophoresis.

51. The method of claim 50 wherein said electrophoresis is carried out in a gel arranged in a slab or arranged in a capillary using a denaturing or non-denaturing medium.

52. The method of claim 1 wherein a predetermined one or more nucleotide sequences in said database are of interest, and wherein the target nucleotide subsequences are such that said sequences of interest are predicted to produce at least one output signal that is not predicted to be produced by other nucleotide sequences in said database.

53. The method of claim 52 wherein the nucleotide sequences of interest are a majority of the sequences in said database.

54. A method for identifying or classifying a nucleic acid in a microsomal sample comprising a plurality of nucleic acids having different nucleotide sequences, said method comprising:
(a) providing a nucleic acid (b) probing said nucleic acid with a plurality of recognition means, each recognition means recognizing a target nucleotide subsequence or a set of target nucleotide subsequences, in order to produce an output set of signals, each signal of said output set representing whether said target nucleotide subsequence or one of said set of target nucleotide subsequences is present in said nucleic acid; and
(c) searching a nucleotide sequence database, said database comprising a plurality of known nucleotide sequences of nucleic acids that may be present in the sample, for sequences predicted to produce said output set of signals, a sequence from said database being predicted to produce an output set of signals when the sequence from said database (i) comprises the same target nucleotide subsequences represented as present, or comprises target nucleotide subsequences that are members of the sets of target nucleotide subsequences represented as present by the output set of signals, and (ii) does not comprise the target nucleotide subsequences not represented as present or that are members of the sets of target nucleotide subsequences not represented as present by the output set of signals,
whereby the nucleic acid is identified or classified.

55. A method for identifying, classifying, or quantifying DNA molecules in a sample of DNA molecules with a plurality of nucleotide sequences, the method comprising the steps of:
(a) providing a CDNA sample synthesized from microsomal RNA molecules;
(b) digesting said sample with one or more restriction endonucleases, each said restriction endonuclease recognizing a subsequence recognition site and digesting DNA to produce fragments with 3′ overhangs;
(c) contacting said fragments with shorter and longer oligodeoxynucleotides, each said longer oligodeoxynucleotide consisting of a first and second contiguous portion, said first portion being a 3′ end subsequence complementary to the overhang produced by one of said restriction endonucleases, each said shorter oligodeoxynucleotide complementary to the 3′ end of said second portion of said longer oligodeoxynucleotide stand;
(d) ligating said longer oligodeoxynucleotides to said DNA fragments to produce a ligated fragments and removing said shorter oligodeoxynucleotides from said ligated DNA fragments;
(e) extending said ligated DNA fragments by synthesis with a DNA polymerase to form blunt-ended double stranded DNA fragments;
(f) amplifying said double stranded DNA fragments by use of a DNA polymerase and primer oligodeoxynucleotides to produce amplified DNA fragments, each said primer oligodeoxynucleotide having a sequence comprising that of a longer oligodeoxynucleotide;
(g) determining the length of the amplified DNA fragments; and
(h) searching a DNA sequence database, said database comprising a plurality of known DNA sequences that may be present in the sample, for sequences predicted to produce one or more of said fragments of determined length, a sequence from said database being predicted to produce a fragment of determined length when the sequence from said database comprises recognition sites of said one or more restriction endonucleases spaced apart by the determined length,
whereby DNA sequences in said sample are identified, classified, or quantified.

56. A method of detecting one or more differentially expressed genes in an in vitro cell exposed to an exogenous factor relative to an in vitro cell not exposed to said exogenous factor comprising:
(a) performing the method of claim 1 wherein said plurality of nucleic acids comprises CDNA of RNA isolated from a microsome of said in vitro cell exposed to said exogenous factor;
(b) performing the method of claim 1 wherein said plurality of nucleic acids comprises cDNA of RNA isolated from a microsome of said in vitro cell not exposed to said exogenous
factor; and
(c) comparing the identified, classified, or quantified cDNA of said in vitro cell exposed to said exogenous factor with the identified, classified, or quantified CDNA of said in vitro cell not exposed to said exogenous factor, whereby differentially expressed genes are identified, classified, or quantified.

57. A method of detecting one or more differentially expressed genes in a diseased tissue relative to a tissue not having said disease comprising:
(a) performing the method of claim 1 wherein said plurality of nucleic acids comprises cDNA of RNA of said diseased tissue, such that one or more CDNA molecules are identified, classified, and/or quantified;
(b) performing the method of claim 1 wherein said plurality of nucleic acids comprises cDNA of RNA of said tissue not having said disease, such that one or more cDNA molecules are identified, classified, and/or quantified; and
(c) comparing said identified, classified, and/or quantified CDNA molecules of said diseased tissue with said identified, classified, and/or quantified CDNA molecules of said tissue not having the disease,
whereby differentially expressed cDNA molecules are detected.

58. The method of claim 57 wherein the step of comparing further comprises determining cDNA molecules which are reproducibly expressed in said diseased tissue or in said tissue not having the disease and further determining which of said reproducibly expressed CDNA molecules have significant differences in expression between the tissue having said disease and the tissue not having said disease.

59. The method of claim 57 wherein said determining cDNA molecules which are reproducibly expressed and said significant differences in expression of said cDNA molecules in said diseased tissue and in said tissue not having the disease are determined by a method comprising applying statistical measures.
